(19) United States
(12) Patent Application Publication
(10) Pub. No.: US 2025/0302308 A1
(43) Pub. Date: Oct. 2, 2025

(54) Methods And Systems For Surgical Navigation Using Spatial Registration Of Tissue Fluorescence During A Resection Procedure

(71) Applicant: Stryker European Operations Limited, Carrigtwohill, Co Cork (IE)

(72) Inventors: Kevin Buckley, Cork (IE); David Eustace, Cork (IE); Stephen Faul, Cork (IE); Gerard W. Nunan, Cork (IE)

(73) Assignee: Stryker European Operations Limited, Carrigtwohill, Co Cork (IE)

(21) Appl. No.: 18/865,607

(22) PCT Filed: May 15, 2023
(86) PCT No.: PCT/IB2023/054995

§ 371 Date: Nov. 13, 2024

Related U.S. Application Data
(60) Provisional application No. 63/364,695, filed on May 13, 2022.

(51) Int. Cl. A61B 5/00

(52) U.S. Cl. CPC A61B 5/0071; A61B 5/0084; A61B 5/742

(57) ABSTRACT

A neurosurgical method for determining a resection status of a tumor is described. The method includes acquiring a medical image of a human organ including a segmented tumor. The method further includes determining a pose of a suction tool including at least one optical fiber and a navigation tracker. The method further includes generating excitation light for the at least one optical fiber to excite the target area which includes the tumor and a margin area surrounding the tumor. The method further includes receiving collected fluorescence emitted from the target area. The method further includes determining whether tissue in the target area corresponds to the tumor based on the collected fluorescence at the pose of the suction tool. The method further includes displaying the resection status of the target area relative to the medical image.

### RELATED APPLICATIONS

This applicant claims priority to and all the benefits of U.S. Provisional Patent Application No. 63/364,695, filed May 13, 2022, the entire contents of which are hereby incorporated by reference.

### BACKGROUND

Glioma tumors may start in the glial cells of the brain or the spine. A surgical procedure, more specifically tumor resection, is often performed to resect the tumor. The goal of a surgical procedure for tumor resection is to achieve gross total resection (GTR). A very aggressive form of glioma is glioblastoma. In patients with glioblastoma, GTR has been shown to prolong the life of a patient. For example, one study showed a 16 months of survival post resection for GTR patients but only 10 months of survival post resection for patients where only 60% of the tumor was resected, resulting in a difference of 60% increase in survival months post resection.

Prior to the resection procedure, a pre-operative image of the patient may be captured by a magnetic resonance imaging (MRI) system. The pre-operative image may be used by a healthcare professional to plan the resection procedure. However, during the resection procedure brain shift (i.e., deformation of the brain) may occur. Brain shift may be caused by a variety of factors such as gravity, head position, fluid drainage, swelling of the brain tissue, tissue manipulation, tissue size, and changes in intracranial pressure caused by the resection of the tumorous tissue or by the craniotomy. In some instances, an intraoperative magnetic resonance image (iMRI) may be captured at the beginning of the resection procedure to account for brain shift occurring after the craniotomy is performed. By capturing an iMRI, brain shift caused by the craniotomy may be captured and accounted for. Subsequent intraoperative iMRIs may be captured throughout the procedure such as after the healthcare professional has completed a portion of the resection procedure to ensure additional brain shift did not occur during resection of the tumorous tissue and after the healthcare professional has completed the resection procedure to confirm that the healthcare professional has achieved GTR. However, iMRI systems may be very costly and capturing each MRI may take anywhere from 30 minutes to 1 hour making capturing multiple iMRIs during a resection procedure cumbersome. Alternatively to performing iMRIs, an ultrasound image may be captured of the tumorous tissue and then related backed to the pre-operative images to account for brain shift. Such ultrasound systems may help to account for brain shift but do not provide any other useful information such as information related to biochemical/cellular information of the tumorous tissue.

Fluorescence guided surgery may be used in patients with high grade glioma. Fluorescence guided surgery improves the chances of achieving GTR. 5-Aminolevulinic Acid (5-ALA) is often given to patients a couple hours before surgery. 5-ALA is a compound that occurs naturally in the hemoglobin synthesis pathway. In cancer cells, the hemoglobin synthesis is disrupted and the pathway stalls at an intermediate compound called Protoporphyrin IX (PpIX). During surgery, the healthcare professional may illuminate an area of brain tissue with excitation light (i.e., blue light) from a surgical microscope. The surgery may be carried out in a darkened or dimmed operating room environment. High-grade tumor cells containing PpIX absorb the excitation light and emit fluorescence (i.e., red fluorescence) having specific optical characteristics. The fluorescence may be observed by the healthcare professional from the surgical microscope.

Fluorescence guided surgery increases the chances of GTR in high-grade tumors such as with glioblastoma tumors. At present, GTR of lower grade tumors is comparatively low because it is difficult to find the margins. 5-ALA cannot be used to improve the outcome of lower-grade tumor resection as the tumor cells only emit a low level of fluorescence and the human eye is not sensitive enough to detect such low levels of fluorescence even with the use of the surgical microscope. The varying fluorescence emitted by cells associated with specific parts of the brain provides rich biochemical/cellular information regarding the cells of the tumorous tissue. Present systems however do not relate the collected fluorescence with the MRI images displayed on the surgical navigation display. Thus, systems and methods are desirable that relate the level of fluorescence to the MRI images in order to account for brain shift, confirm GTR, and provide biochemical/cellular information regarding the cells associated with the tumorous tissue.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

In a feature, a neurosurgical method for determining a resection status of a tumor during a resection procedure is described. The method includes acquiring at least one medical image of a human organ including a segmented tumor. The method determining a pose of a suction tool, including at least one optical fiber and a navigation tracker, based on the navigation tracker. The method includes generating excitation light for the at least one optical fiber to excite a target area of the human organ. The target area including the tumor and a margin area surrounding the tumor. The method includes receiving collected fluorescence emitted from the target area from the at least one optical fiber. The method includes determining whether tissue in the target area corresponds to the tumor based on the collected fluorescence at the pose of the suction tool. The method includes displaying the resection status of the target area relative to the at least one medical image based on the determination of whether the tissue corresponds to the tumor and the pose of the suction tool.

In a feature, a neurosurgical system for determining a resection status of tumorous tissue of a target area is described. The neurosurgical system includes a suction tool, a navigation tracker, an optical fiber, an excitation source, an optical instrument, and a surgical navigation system. The suction tool is configured to apply suction to a brain tissue of a patient. The suction tool includes a suction cannula defining a lumen. The navigation tracker is coupled to the suction tool. The optical fiber is coupled to the suction cannula. The optical fiber being configured to transmit a fluorescence emitted by the brain tissue. The excitation source is configured to emit an excitation light having a wavelength to induce the fluorescence of the tumorous tissue. The optical instrument is coupled to the optical fiber. The optical instrument is configured to convert the fluorescence emitted by the brain tissue and transmitted by the optical fiber into an electrical signal. The surgical navigation system is configured to receive at least one medical image of a human organ including a segmented tumor. The surgical navigation system is also configured to determine a pose of the suction tool based on the navigation tracker. The surgical navigation system is also configured to determine whether tissue in the target area corresponds to the tumorous tissue based on the collected fluorescence at the pose of the suction tool. The surgical navigation system is also configured to display at least one indicator relative to the at least one medical image based on the determination of whether the tissue in the target area corresponds to the tumorous tissue and the pose of the suction tool.

In a feature, a neurosurgical method for determining resection status for a tumor from a human organ during a resection procedure is described. The neurosurgical method includes navigating a suction tool including a navigation tracker within the human organ to a target area corresponding to a segmented tumor of at least one medical image. The method includes determining a pose of the suction tool based on the navigation tracker. The method includes applying excitation light, with an optical fiber coupled to the suction tool, the optical fiber being connected to an excitation source, to the target area. The method includes removing tissue from the target area with the suction tool while collecting fluorescence from the target area with the optical fiber coupled to an optical instrument, the target area including the tumorous tissue and a margin area surrounding the tumorous tissue. The method includes viewing at least one virtual indicator overlaid onto at least one medical image of the human organ including a segmented target area based on the pose of the suction tool in response to a surgical navigation system connected with the optical instrument determining that the tissue corresponds to the tumor. The method includes comparing the at least one virtual indicator to a shape of the segmented target area to determine whether any residual tumor remains.

In a feature, a neurosurgical method for determining an extent of tumorous matter removed from a human organ is described. The neurosurgical method includes the acquiring at least one medical image of the human organ including a segmented tumor. The method includes navigating a surgical tool including a navigation tracker and at least one optical fiber within the human organ to a target area corresponding to the segmented tumor of the at least one images. The method includes determining a pose of the surgical tool based on the navigation tracker. The method includes determining whether tissue of the target area is tumorous at the determined pose of the surgical tool based on fluorescence emitted from the tissue. The method includes displaying with the surgical navigation system (i) a first indicator overlaid onto the at least one medical image of the human organ based on the pose of the surgical tool in response to the step of determining that the tissue is tumorous and (ii) a second indicator overlaid onto the at least one medical image of the human organ based on the pose of the surgical tool in response to the step of determining that the tissue is not tumorous.

In a feature, a neurosurgical system for determining an extent of tumorous matter removed from a human organ is described. The neurosurgical system includes a surgical tool, an optical system, and a surgical navigation system. The surgical tool system includes a surgical tool with a navigation tracker disposed on the surgical tool. The surgical tool is configured to remove tissue from a target area of the human organ. The optical system includes at least one optical fiber, the at least one optical fiber being coupled to the surgical tool, the at least one optical fiber is configured to illuminate excitation light at the target area and collect fluorescence emitted from the target area. The optical system being configured to convert the fluorescence into an electrical signal. The surgical navigation system is configured to: receive at least one medical image of the human organ including a segmented tumor. The surgical navigation system is configured to determine a pose of the surgical tool based on the navigation tracker. The surgical navigation system is configured to determine whether tissue of the target area is tumorous at the determined pose of the surgical tool based on the electrical signal. The surgical navigation system is configured to display (i) a first indicator overlaid onto the at least one medical image of the human organ based on the pose of the surgical tool in response to determining that the tissue is tumorous and (ii) a second indicator overlaid onto the at least one medical image of the human organ based on the pose of the surgical tool in response to determining that the tissue is not tumorous.

In a feature, a neurosurgical method for determining a resection status of a tumor during a resection procedure is described. The method includes acquiring at least one medical image of a human organ including a segmented tumor. The method includes determining a pose of a suction tool, including at least one optical fiber and a navigation tracker, based on the navigation tracker. The method includes generating excitation light for the at least one optical fiber to excite a target area of the human organ, the target area including the tumor and a margin area surrounding the tumor. The method includes receiving collected fluorescence emitted from the target area from the at least one optical fiber. The method includes determining an intensity of the collected fluorescence. The method includes generating a point cloud based on the intensity of the collected fluorescence and the pose of the suction tool.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 depicts a neurosurgical system according to the teachings of the present disclosure.

FIG. 2 depicts a functional block diagram of a neurosurgical system according to the teachings of the present disclosure.

FIG. 3 depicts an example suction tool of a neurosurgical system according to the teachings of the present disclosure.

FIG. 4 depicts a functional block diagram of a tissue detection system of a neurosurgical system according to the teachings of the present disclosure.

FIG. 5 depicts a tissue detection system of a neurosurgical system according to the teachings of the present disclosure.

FIGS. 6A and 6B depict an optical system of a tissue detection system of a neurosurgical system according to the teachings of the present disclosure.

FIGS. 7A and 7B depict an exploded view of several of the components of the optical system of a tissue detection system of a neurosurgical system according to the teachings of the present disclosure.

FIG. 8 depicts a sample element coupled to a suction tool with a jacket removed of a neurosurgical system according to the teachings of the present disclosure.

FIG. 9 depicts a sample element coupled to a suction tool of a neurosurgical system according to the teachings of the present disclosure.

FIG. 10 depicts a graphical user interface of a navigation computer of a neurosurgical system, the graphical user interface including multiple views of a brain of a patient according to the teachings of the present disclosure.

FIGS. 11A-11F depict a graphical user interface of a navigation computer of a neurosurgical system, the graphical user interface including an axial view of a brain of a patient according to the teachings of the present disclosure.

FIGS. 12A-12E depict a graphical user interface of a navigation computer of a neurosurgical system, the graphical user interface including an axial view of a brain of a patient according to the teachings of the present disclosure.

FIGS. 13A and 13B depict a graphical user interface of a navigation computer of a neurosurgical system, the graphical user interface including an axial view of a brain of a patient according to the teachings of the present disclosure.

FIG. 14 depicts a graphical user interface of a navigation computer of a neurosurgical system, the graphical user interface including an axial view of a brain of a patient according to the teachings of the present disclosure.

FIG. 15 depicts a graphical user interface of a navigation computer of a neurosurgical system, the graphical user interface including a three-dimensional (3D) point cloud of resected tissue, a three-dimensional (3D) model of resected tumorous tissue, and a three-dimensional (3D) model of tumorous tissue according to the teachings of the present disclosure.

FIG. 16 depicts an exemplary method performed by a neurosurgical system according to the teachings of the present disclosure.

FIG. 17 depicts an exemplary method performed by a neurosurgical system according to the teachings of the present disclosure.

In the drawings, reference numbers may be reused to identify similar and/or identical elements.

### DETAILED DESCRIPTION

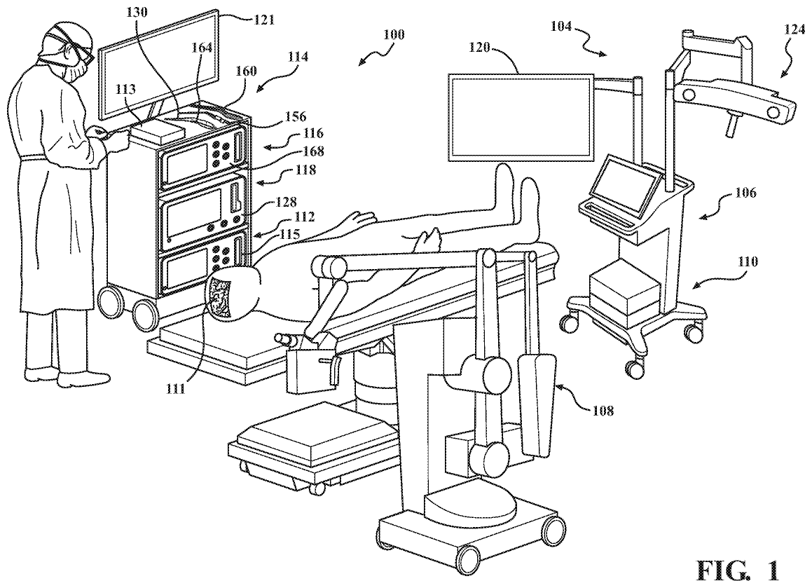

With reference to FIG. 1, the neurosurgical system 100 may include a surgical navigation system 104, a surgical microscope 108, a surgical cart 114, and a suction system 113. The surgical navigation system 104 includes a cart assembly 106 that houses a navigation computer 110. The navigation computer 110 may also be referred to as the navigation controller. A navigation interface is in operative communication with the navigation computer 110. The navigation interface may include one or more displays 120. The navigation interface may include one or more input devices which may be used to input information into the navigation computer 110 or otherwise to select/control certain aspects of the navigation computer 110. Such input devices may include interactive touchscreen displays/menus, a keyboard, a mouse, a microphone (voice-activation), gesture control devices, or the like.

The navigation computer 110 may be configured to store one or more pre-operative or intra-operative images of the brain. Any suitable imaging device may be used to provide the pre-operative or intra-operative images of the brain. For example, any 2D, 3D or 4D imaging device, such as isocentric fluoroscopy, bi-plane fluoroscopy, ultrasound, computed tomography (CT), multi-slice computed tomography (MSCT), magnetic resonance imaging (MRI), positron emission tomography (PET), optical coherence tomography (OCT). The images may also be obtained and displayed in two, three or four dimensions. In more advanced forms, four-dimensional surface rendering regions of the body may also be achieved by incorporating patient data or other data from an atlas or anatomical model map or from pre-operative image data captured by MRI, CT, or echocardiography modalities. As the phrase one or more images will be referred to throughout the disclosure, it is understood that the phrase may refer to the pre-operative images or the intra-operative images captured during the resection procedure.

The navigation computer 110 may generate the one or more images of the brain on a display 120. The navigation computer 110 may also be connected with the surgical microscope 108. For example, the display 120 may show an image corresponding to the field of view of the surgical microscope 108. The navigation computer 110 may include more than one display, with one such display showing the field of view of the surgical microscope 108 while the other such display may show the one or more images of the brain.

The tracking system 124 may be an optical tracking system and may be coupled to the navigation computer 110. The tracking system 124 is configured to sense the pose (i.e., position and orientation) of a navigation tracker attached to or integrated with each of one or more of the various surgical tools described herein (e.g., suction tool 156, bipolar forceps 160, ultrasonic handpiece assembly 130), and provide the pose to the navigation computer 110 to determine a pose of the surgical tool, such as relative to a target area of the patient, as discussed in greater detail below. Each navigation tracker may include one or more tracking elements, which may be active or passive infrared tracking elements detectable by a camera of the optical tracking system. An example of a surgical navigation system 104 which includes a tracking system is Nav3i™ that is commercially available from Stryker. The surgical navigation system 104 may have various functions and features as described in U.S. Pat. No. 7,725,162 B2 and U.S. Pat. Pub. No. 2020/0100849 A1 which are hereby incorporated by reference in their entireties. While the example is provided that the tracking system 124 is an optical tracking system, other tracking systems may be employed.

For instance, in some implementations, the tracking system 124 may be realized as an electromagnetic tracking system, with each navigation tracker including a position sensor located at and/or embedded within the distal end of one of the various surgical tools that enables the distal end of the surgical tool to be to be tracked, such as relative to a target area of the patient. More specifically, the position sensor may include a coil that is in communication with one or more electrical conduits extending along the length of the surgical tool. When position sensor, or more particularly the coil, is positioned within an electromagnetic field, movement of position sensor within that magnetic field may generate electrical current in the coil, which may then be communicated along the electrical conduits to the navigation computer 110. This phenomenon may enable the navigation computer 110 to determine the location of distal end of the surgical tool within a three-dimensional space, such as relative to a target area of patient tissue.

By way of example only, position sensor may be constructed and operable in accordance with at least some of the teachings of U.S. Pat. No. 8,702,626, the disclosure of which is incorporated by reference herein; U.S. Pat. No. 8,320,711, the disclosure of which is incorporated by reference herein; U.S. Pat. No. 8,190,389, the disclosure of which is incorporated by reference herein; U.S. Pat. No. 8,123,722, the disclosure of which is incorporated by reference herein; U.S. Pat. No. 7,720,521, the disclosure of which is incorporated by reference herein; U.S. Pat. Pub. No. 2014/0364725, the disclosure of which is incorporated by reference herein; U.S. Pat. Pub. No. 2014/0200444, the disclosure of which is incorporated by reference herein; U.S. Pat. Pub. No. 2012/0245456, the disclosure of which is incorporated by reference herein; U.S. Pat. Pub. No. 2011/0060214, the disclosure of which is incorporated by reference herein; U.S. Pat. Pub. No. 2008/0281156, the disclosure of which is incorporated by reference herein; and/or U.S. Pat. Pub. No. 2007/0208252, the disclosure of which is incorporated by reference herein.

The surgical microscope 108 includes one or more objectives configured to provide magnification in a range (e.g., from about 2 times to about 50 times). The surgical microscope 108 can have a field of view having an area of a predetermined range. The surgical microscope 108 is configured for fluorescence microscopy, for example, to detect PpIX. The surgical microscope 108 may include one or more excitation sources (e.g., an excitation source configured to emit light in the visible light spectrum or an excitation source configured to emit light in the infrared spectrum) for illuminating the brain tissue 111 with excitation light to cause the PpIX to fluorescence. The surgical microscope 108 may also include a camera capable of detecting radiation at the fluorescent wavelengths of PpIX or ICG.

The surgical cart 114 may include a surgical system 112, a suction system 113, a tissue detection system 116, and an ultrasonic surgical system 118. A display 121 may be coupled to the surgical cart and operatively connected to the surgical system 112, the tissue detection system 116, and/or the ultrasonic surgical system 118 to display information related with each respective system 112, 116, and 118.

The suction tool 156 may be connected to the suction system 113 via a suction tube. The suction system 113 may include one or more containers for storing the waste collected by the suction tool 156. The suction system 113 may receive suction from a vacuum source, such as a vacuum outlet of a medical facility. The suction system 113 may include one or more regulators or one or more adjustment valves for controlling the suction pressure received from the vacuum source. The one or more regulators or one or more adjustment valves may be omitted, and the suction tube may be directly or indirectly connected via the one or more containers to the vacuum outlet. In an example, the suction system 113 may correspond to a wall suction unit. In another example, the suction system 113 may correspond to a portable suction unit. The suction system 113 and the suction tool 156 may have various features, as described in U.S. Pat. No. 9,066,658 and U.S. Pat. Pub. No. 20180344993 which are hereby incorporated herein by reference in their entireties.

The surgical system 112 may include a surgical tool, such as bipolar forceps 160, and a surgical control console 115 to control various aspects of the surgical tool. For example, the surgical system 112 may be configured to control electric current output by the system. The healthcare professional may also use the surgical tool to perform any surgical operation on the tissue. For example, to ablate the tissue or to cauterize the tissue. The bipolar forceps may have features, as described in U.S. Pat. No. 8,361,070 B2 which is hereby incorporated by reference in its entirety. While the disclosure discusses and illustrates that the surgical tool may include bipolar forceps 160, the surgical system 112 and surgical tool may include other tools, such as a neuro stimulator, a dissector, or an ablation device (e.g., an RF ablation device and/or a laser ablation device). For example, the surgical system and/or surgical tools may have various features as described in U.S. Pat. No. 8,267,934 B2 which is hereby incorporated by reference in its entirety. Any number of surgical systems and any number of surgical tools may be employed by the healthcare professional in performing the surgical procedure.

The ultrasonic surgical system 118 may include an ultrasonic control console 128 and an ultrasonic handpiece assembly 130 used by a healthcare professional to ablate the brain tumor. The ultrasonic control console 128 may also be configured to provide irrigation and/or aspiration via one or more tubes (not shown) connected to the ultrasonic handpiece assembly 130 and regulate the irrigation and/or aspiration functions of the ultrasonic handpiece assembly 130 to optimize performance of the ultrasonic handpiece assembly 130. The ultrasonic handpiece assembly 130 may have various features, as described in U.S. Pat. Nos. 6,497,715 B2; 6,955,680 B2; and 6,984,220 B2 and PCT Publication WO 2020/068756 A1; which are hereby incorporated herein by reference in their entireties. An example of ultrasonic surgical systems that may be used are commercially available from Stryker including Sonopet IQ Ultrasonic Aspirator. The ultrasonic control console 128 may control various operation parameters based on signals received from the tissue detection system 116.

The tissue detection system 116 may include a control console 168 and a sample element 164. The control console 168 may generate a real-time indication which is viewable within the sterile field via the sample element 164 when brain tissue 111 corresponds to tumorous tissue. The sample element 164 may also be coupled to the bipolar forceps 160, the suction tool 156, or other surgical tools as will be described in greater detail below. The tissue detection system 116 determines when the brain tissue 111 corresponds to tumorous tissue based on fluorescence emitted by the target tissue caused by the fluorophore. In an example, the fluorophore may correspond to PpIX. In another example, the fluorophore may correspond to ICG. As will be discussed in greater detail below, based on the intensity and the wavelengths of the fluorescence emitted by PpIX, the tissue detection system 116 may determine that the tumorous tissue is present.

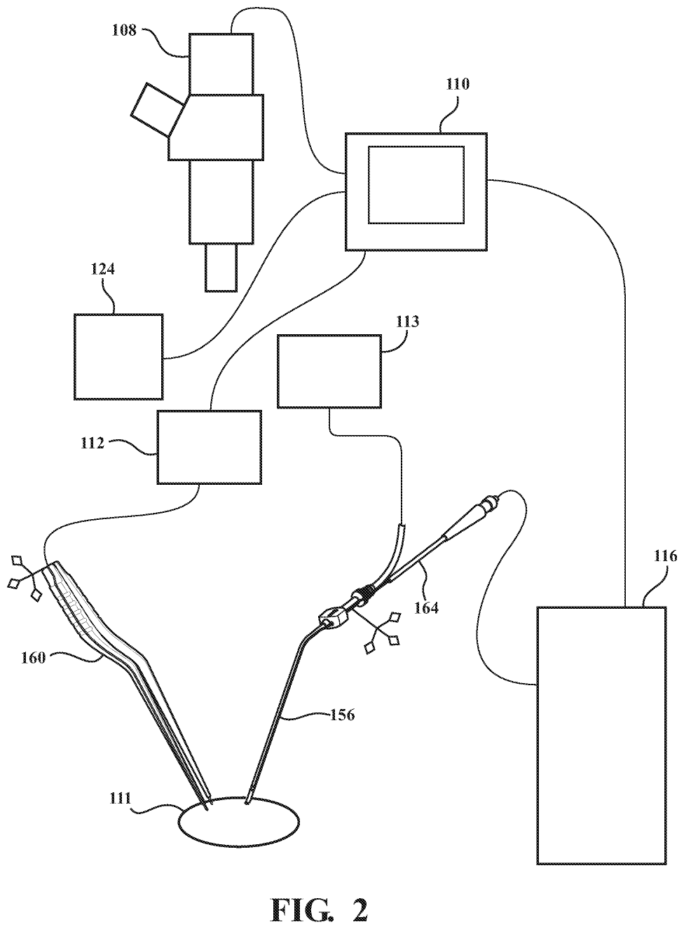

With reference to FIG. 2, a schematic of the neurosurgical system 100 is shown. The tissue detection system 116 allows the healthcare professional to detect the presence of PpIX in real-time and may be used in conjunction with the surgical microscope 108 to improve the outcome of a tumor resection procedure and the chances of achieving GTR. During the surgical procedure, the healthcare professional may initially view the brain tissue 111 of the patient with the surgical microscope 108 under excitation light (e.g., the blue light) to identify which portion of the brain tissue 111 corresponds to the target tissue evidenced by the red fluorescence. The healthcare professional may switch the surgical microscope 108 back to standard white light illumination for better visibility and begin resection of the target tissue. Since the sample element 164 may be coupled to the suction tool 156, the healthcare professional does not have to account for any additional surgical tools (i.e., optical probes or the like) in the sterile field. The healthcare professional may perform the resection of the target tissue with the bipolar forceps 160 in the one hand and the suction tool 156 in the other hand.

As the healthcare professional is resecting the target tissue, the control console 168 may function to provide the healthcare professional with a real-time indication of the target tissue in the brain tissue 111 by activation of an indicator (discussed in greater detail below) of the sample element 164. The tissue detection system 116 according to the teachings of the present disclosure prevents the healthcare professional from having to switch back and forth between the various illumination settings of the surgical microscope 108 (i.e., illuminating the tissue with excitation light and white light) as the healthcare professional is performing resection of the target tissue. This becomes especially beneficial as the healthcare professional approaches the margin of the target tissue because it is desirable for the healthcare professional to achieve GTR but to leave as much healthy tissue intact as possible.

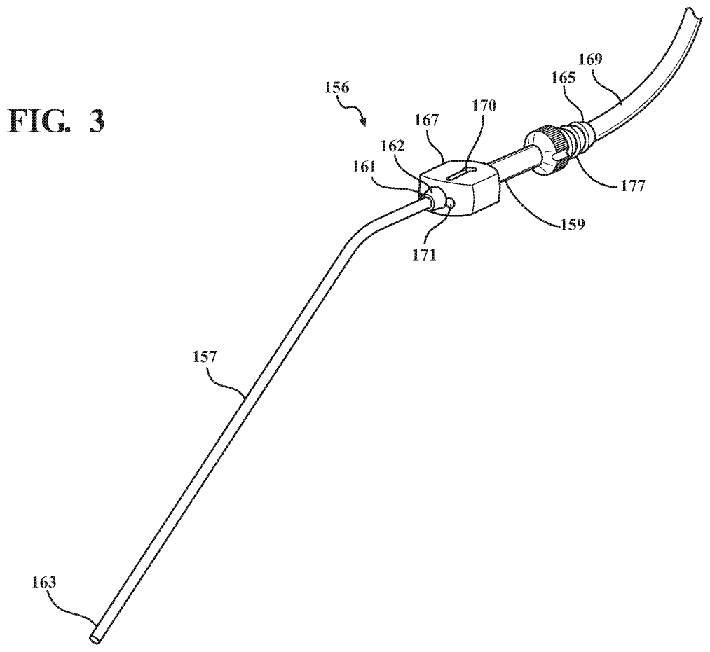

With reference to FIG. 3, the suction tool includes a suction cannula 157 and a handle 159. The suction cannula 157 defines a lumen for suctioning fluid, debris, and tissue from a patient. The handle 159 is tubular shaped with a control portion 167. A distal end 162 of the handle 159 (or a distal end 162 of the control portion 167) may be tapered and is configured to receive a proximal end 161 of a suction cannula 157. A proximal end 165 of the handle 159 includes a vacuum fitting which may be configured to receive a suction tube 169 which is connected to the vacuum source which generates the suction pressure. The vacuum fitting may be a standard barbed fitting, quick disconnect, or any other suitable fitting known in the art to allow the suction tube to be fluidly coupled to a vacuum source.

The control portion 167 may include a teardrop shaped control 170 for regulation of suction pressure. For example, when no portion of the teardrop shaped control 170 is covered by the healthcare professional, suction pressure may be minimal, and when the teardrop shaped control 170 is covered completely, suction pressure may be at its maximum. While the control portion 167 is described as including a teardrop shaped control, the control portion 167 may include another suitable input such as a button or different shaped control to allow the healthcare professional to vary the suction pressure. The control portion 167 may include a through bore 171 for receiving the sample element 164, as will be discussed in greater detail below. The healthcare professional holds the suction tool 156 from its handle 159, manipulating the suction tool 156 so that the distal end 163 contacts the tissue of the patient during the surgical procedure in order to provide suction at the desired location. While the suction tool 156 is described as having a Fukushima configuration, other configurations are contemplated such as a Frazier or Poole configuration.

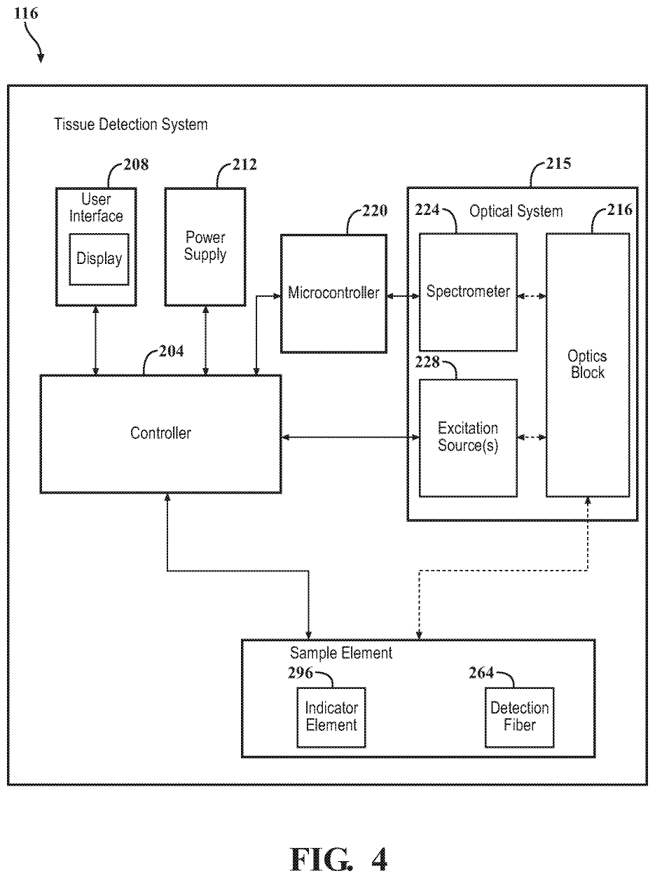

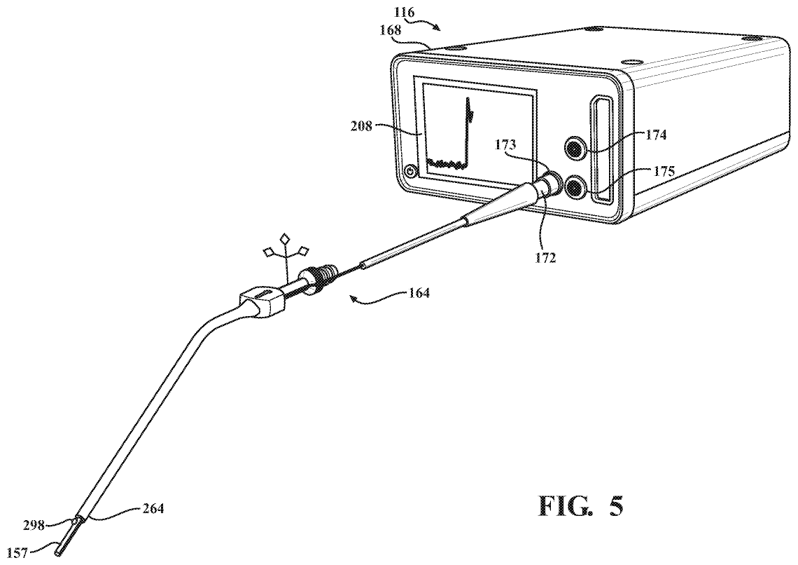

With reference to FIGS. 4 and 5, the tissue detection system 116 includes the sample element 164 and a control console 168. As shown, the sample element 164 may be coupled to the suction tool 156. The sample element 164 may be connected to the control console 168 via connector 172. The sample element 164 may include a detection fiber 264 and an indicator element 296, as discussed in greater detail below. The control console 168 may include a controller 204, a user interface 208, a power supply 212, an optical system 215, and a microcontroller 220. The optical system 215 may include an optics block 216, a spectrometer 224, an excitation source 228, and an optical connector 229. The function of each component will be discussed in greater detail below.

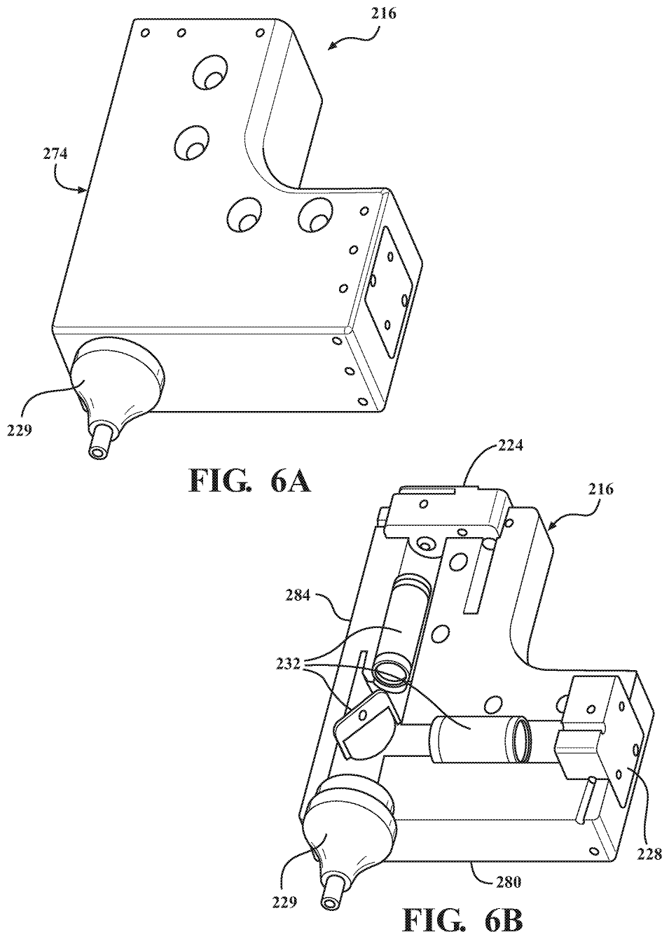

The user interface 208 may include a display for displaying output from the controller 204 related to the fluorescence collected from the tissue. The user interface 208 may also include one or more inputs (e.g., a push button, a touch button, a switch, etc.) configured for engagement by the healthcare professional. The power supply 212 may supply power to various components of the control console 168. The control console 168 may include a probe port 173 in which the connector 172 of the sample element 164 is connected. The detection fiber 264 may then be connected to the optics block 216 via the optical connector 229, an example of which is illustrated in FIGS. 6A and 6B. The control console 168 may also include an electrical port 174 for establishing communication links, such as to the surgical system 112 and the ultrasonic surgical system 118. The communication links may also be established wirelessly.

The excitation source 228 may generate excitation light to be illuminated at the target tissue by the healthcare professional via the detection fiber 264. The excitation source 228 may be configured to emit the excitation light within a predetermined wavelength range (e.g., blue light at about 405 nm or blue light in the range of 400 nm to 500 nm). The excitation source 228 may also be configured to emit excitation light corresponding to other wavelengths such as wavelengths associated with the rest of the visible light spectrum other than blue light (e.g., greater than 500 nm but less than 700 nm), and wavelengths associated with the ultraviolet light spectrum (less than 400 nm) and/or infrared light spectrum (greater than 700 nm). The excitation source 228 may include any number of light sources such as a light emitting diode (LED), a pulsed laser, a continuous wave laser, a modulated laser, a filtered white light source, etc.

The system may include other excitation sources that may be further configured to emit excitation light corresponding to different wavelengths other than as described above. In this implementation, the excitation source may be referred to as a first excitation source 228 configured to emit a first excitation light within a first predetermined wavelength range of the visible light spectrum, and a second excitation source may be configured to emit infrared light within a second wavelength range corresponding to the infrared light spectrum (e.g., 700 nm to 1 mm). The first excitation source 228 may be configured to emit light which would excite a first fluorophore such as PpIX, while the second excitation source is configured to emit light which would excite a second fluorophore such as ICG.

The controller 204 may control operation of the excitation source 228 by varying operating parameters of the excitation source 228. The operating parameters may correspond to a time setting, a power setting, or another suitable setting. The time setting may include a pulse width. The pulse width may be based on the integration time of the spectrometer 224. The integration time of the spectrometer 224 is discussed in greater detail below.

The detection fiber 264 may be coupled to the optical connector 229. When the sample element 164 is coupled to the suction tool 156, the distal end 272 of the detection fiber 264 is adjacent to the working portion of the surgical tool and allows for the excitation light to be delivered to the target tissue.

With reference to FIGS. 6A and 6B, the optics block 216 is shown. The optical connector 229 may be coupled to the optics block 216. The optics block 216 may include an outer casing 274 constructed of metal or another suitable material and may fully enclose components 232 of the optics block 216. The optics block 216 may be L-shaped and include a first portion 280 and a second portion 284. The excitation source 228 may be coupled to the first portion 280 of the optics block 216. The spectrometer 224 may be coupled to the second portion 284 of the optics block 216.

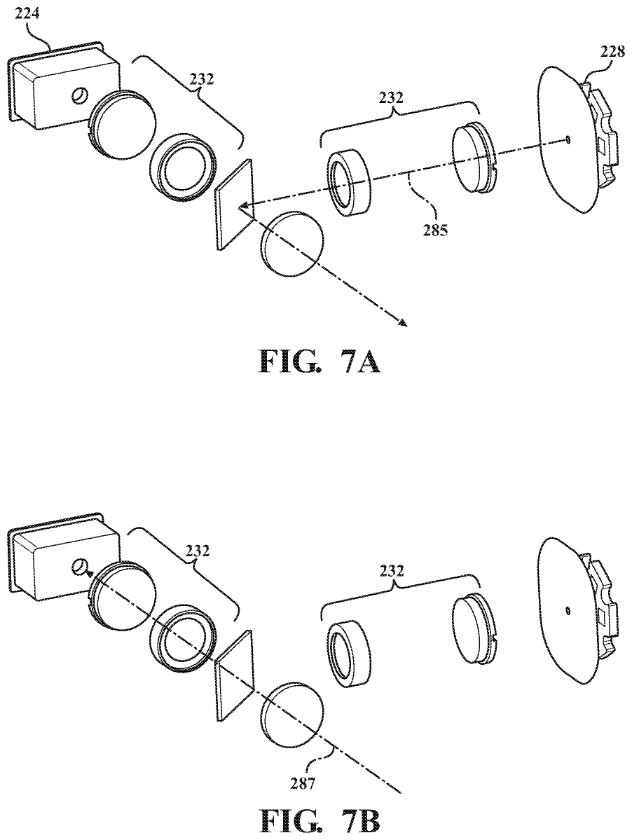

With additional reference to FIGS. 7A and 7B, an exploded view of the components 232 of the optical system 215 is shown illustrating an optical path 285 for the excitation light and the optical path 287 for light collected from the brain tissue 111. The first portion 280 may include the optical path 285 for the excitation light to travel from the one or more excitation sources 228 to the brain tissue 111 via the detection fiber 264. The optical path 285 may be defined by the components 232 in the first portion 280 of the optics block 216. The second portion 284 may include the optical path 287 for the collected light to travel from the brain tissue 111 via the detection fiber 264 to the spectrometer 224. The optical path 287 may be defined by the components 232 in the second portion 284 of the optics block 216. The components 232 of the optics block 216 may include optical components such as one or more laser line filters and one or more long-pass filters. The optics block 216 may include other optical components such as one or more mirrors, lenses, optical connectors, optical fiber, and/or any other suitable optical components.

In FIG. 7A, the excitation source 228 emits the excitation light which travels through one or more components 232, such as a laser line filter and/or bandpass filter. The laser line filter or bandpass filter may be configured to reject unwanted noise (e.g., lower level transitions, plasma, and glows) generated by the excitation source 228. Stated differently, the laser line filter may be configured to clean up the excitation light or make the excitation light more monochromatic. The long-pass filter may be configured to reflect the light down the detection fiber 264 and to the brain tissue 111. The excitation source 228 may be configured to deliver unfiltered excitation light (i.e., the filters may be omitted) via the detection fiber 264 to the target tissue. The detection fiber 264 may guide the excitation light to the brain tissue 111 via the sample element 164.

The detection fiber 264 may be configured to collect light (i.e., fluorescence and ambient light) from the brain tissue 111. The coupling of the sample element 164 to the surgical tool results in the distal end 272 being adjacent to the working portion of the surgical tool as to allow for the light to be collected from the target tissue. Due to the presence of ambient light and/or background light caused by various sources in the operating room such as the surgical microscope 108, surgical lamps, or any other devices in the operating room, the light collected from the brain tissue 111 may include the ambient light and/or background light. With reference to FIG. 7B, the light collected by the detection fiber 264 passes through the components 232, such as the long pass filter, of the second portion 284 of the optics block 216. After the light passes through the components 232, the light may enter the spectrometer 224 which is coupled to the optics block 216.

The detection fiber 264 may be coupled to the optical connector 229. As discussed in greater detail below, the distal end 272 of the detection fiber 264 may include a lens or other transparent material such that when the sample element 164 is positioned on a surgical tool (i.e., the ultrasonic handpiece, the suction tool or the bipolar forceps or other working surgical tool) the coupling of the sample element 164 to the surgical tool results in the distal end 272 of the detection fiber 264 being adjacent to the working portion of the surgical tool as to allow for the excitation light to be delivered to the target tissue.

The spectrometer 224 may be configured to convert the filtered optical light into spectral signals in the form of electrical signals, which may be representative of the fluorescence collected from tissue of the target area when the target area is excited by excitation light. The microcontroller 220 is configured to control operation of the spectrometer 224. Examples of spectrometer systems that may be used are commercially available from Hamamatsu including Mini-spectrometer micro series C12880MA. Although a spectrometer 224 is contemplated throughout the disclosure, other optical instruments may be used instead of a spectrometer 224.

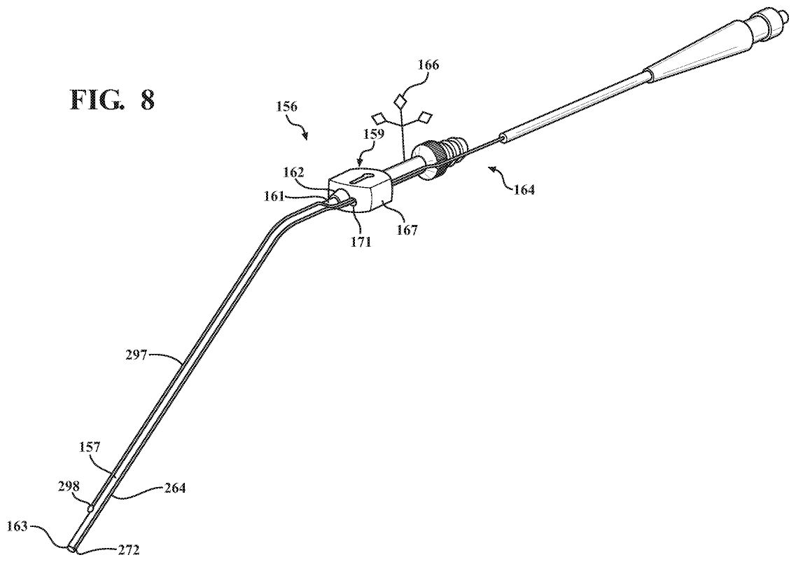

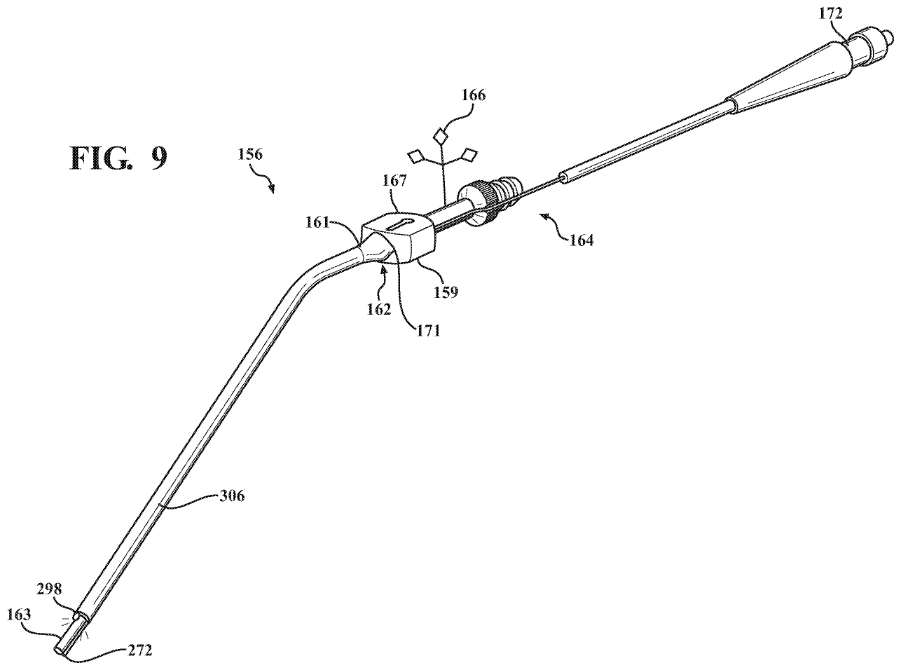

With reference to FIG. 8, the sample element 164 and tracking elements 166 are shown coupled to the suction tool 156. The tracking elements 166 are shown coupled to the handle 159 of the suction tool 156 but may be coupled to any portion of the suction tool 156. The tracking elements 166 may also be coupled to a portion of the sample element 164. The indicator element 296 may include a transmission member 297 connected to an indicator light 298. The indicator light 298 may include one or more light emitting diodes or another suitable light source. The indicator light 298 may be configured to emit light based on an activation signal received from the controller 204. The controller may be configured to generate the activation signal in response to detection of tumorous tissue by the controller 204. The indicator light 298 may be sphere shaped, dome shaped, cylinder shaped, or another suitable shape. A jacket 306 may enclose part of the detection fiber 264 and part of the indicator element 296, specifically the transmission member 297. As shown in FIG. 9, the jacket 306 does not cover the distal end 272 of the detection fiber 264 or the indicator light 298. The jacket 306 may be made from any one of polyvinyl chloride, polyethylene, chlorinated polyethylene, chlorosulfonated polyethylene/neoprene and/or another suitable material.

The detection fiber 264 and a portion of the indicator element 296, (i.e., the transmission member 297 and indicator light 298) may be guided through the through bore 171 of the handle 159. A distal end 272 of the detection fiber 264 may be positioned proximally to a distal end 163 of the suction cannula 157. The indicator light 298 may be positioned near the distal end of the detection fiber 264 but more proximal to a distal end 162 of the handle 159 (or a distal end 162 of the control portion 167) than the distal end 272 of the detention fiber is positioned. In other words, the distal end 272 of the detection fiber 264 may be disposed more proximal to the distal end 163 of the suction cannula 157 than the indicator light 298 is. With additional reference to FIG. 9, after the detection fiber 264 and the portion of the indicator element 296 is fed through the through bore 171, a jacket 306 may be fitted overtop of the suction cannula 157, the detection fiber 264, and the transmission member 297. The jacket 306 may be mated to the distal end 162 of the control portion 167 so that the distal end 162 and the through bore 171 are covered. The jacket 306 may terminate just before where the indicator light 298 is coupled to the suction cannula 157. The detection fiber 264 may protrude from beneath the jacket 306 so that the jacket 306 does not interfere with the delivery of excitation light or collection of fluorescence from the tissue. Also as shown, the indicator light 298 is exposed fully but may be partially covered by the jacket 306. In some configurations, the jacket 306 may be omitted.

Although, the sample element 164 is shown coupled to the suction tool 156, the sample element 164 may be coupled to another surgical tool (e.g., the ultrasonic handpiece assembly 130, the bipolar forceps 160, etc.). The distal end 272 of the detection fiber 264 may include a lens, a collimator, or another suitable optical component that allows the detection fiber 264 to deliver excitation light to the brain tissue 111 and to collect light from the brain tissue

As previously discussed, the detection fiber 264 may carry the excitation light from the optical system 215 to the brain tissue 111 and the detection fiber 264 may also collect light from the brain tissue 111 and deliver the light to the optical system 215. While the example is provided that the detection fiber 264 functions to deliver excitation light to the tissue and also collect light from the tissue, the system may include two separate fibers such as a collection fiber and an excitation fiber instead. The collection fiber may collect light from the tissue and the excitation fiber may deliver excitation light to the tissue. While the detection fiber 264 is contemplated as a single fiber for simplicity, it is understood that the detection fiber 264 may include more than one fiber. For example, the detection fiber 264 may include a bundle of detection fibers all being connected in similar fashion to the single fiber connection discussed above. Further, the detection fiber 264 may include any number of fibers connected in series.

The controller 204 may be configured to utilize the spectral signals provided by the microcontroller 220 to determine or detect one or more properties of collected fluorescence represented by the signals, and to determine or detect the presence of tumorous tissue. The controller 204 may apply or utilize any suitable algorithm or combination of algorithms to detect the presence of tumorous tissue based on the fluorescence intensity of the PpIX determined from the spectral signals. Example algorithms are as disclosed in PCT Application PCT/IB2022/052294, the contents which are herein incorporated by reference. Based on the detection of tumorous tissue or the fluorescence intensity, the controller 204 may provide a healthcare professional with an indication that tumorous tissue has been detected.

The controller 204 may activate the indicator light 298 in response to the detection of the target tissue. The indicator light 298 may emit light when activated to signal to the healthcare professional that the tumorous tissue has been detected. The controller 204 may control the LED or other light source to emit various colors of light depending on whether the controller 204 detects PpIX or ICG (i.e., whether the brain tissue 111 corresponds to the target tissue or a blood vessel). For example, the controller 204 may control the LED to emit green light (e.g., wavelengths of about 520-564 nm) when PpIX above a threshold is detected or yellow light (e.g., wavelengths 565-590 nm) when ICG is detected.

The controller 204 may be configured to communicate with the navigation computer 110 or any other system (e.g., the surgical system 112, the ultrasonic surgical system 118, etc.) of the neurosurgical system 100 via the communication link established through the electrical port 174. For example, a cord may be plugged into the electrical port 174 and also plugged into the navigation computer 110 to establish the communication link. The communication link may also be established wirelessly. The controller 204 may provide the spectral signals, a determination of the level of fluorescence detected, and/or a determination of whether tissue corresponds to healthy tissue or tumorous tissue to navigation computer 110.

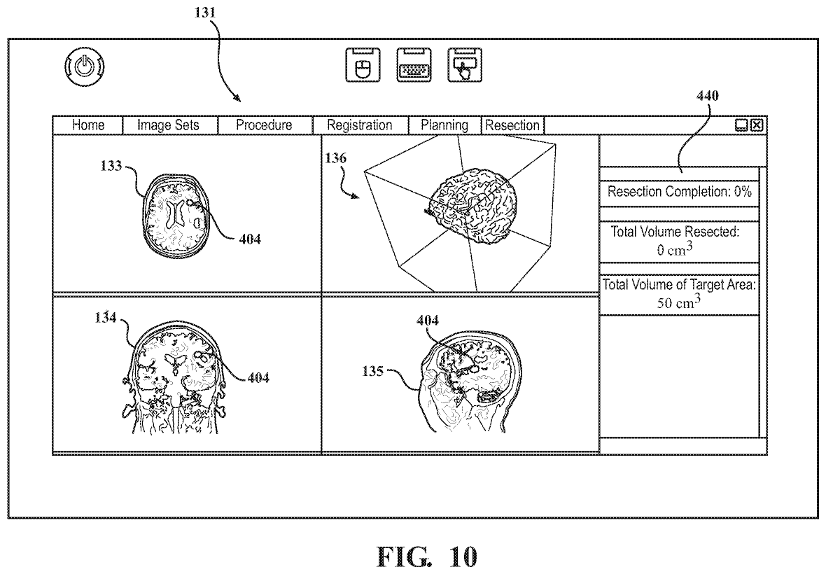

With reference to FIG. 10, the navigation computer 110 may be configured to display graphical user interface (GUI) 131 with an axial view 133 of the brain tissue 111 including the tumorous tissue, a coronal view 134 of the brain tissue 111 including the tumorous tissue, a sagittal view 135 of the brain tissue 111 including the tumorous tissue, and a 3D model 136 of the brain tissue including the tumorous tissue. The navigation computer 110 may be configured to display a pose of one or more of the surgical instruments, such as the suction tool 156 and the bipolar forceps 160, relative to a target area of the images based on the tracking information received from the tracking system 124. The navigation computer 110 may be configured to segment the tumorous tissue of the images using any suitable segmentation technique or combination of segmentation techniques, for example, an automatic segmentation technique, a semi-automatic segmentation technique or a manual segmentation technique. The automatic or semi-automatic segmentation techniques may employ any suitable segmentation method, for example, a region growing method, a watershed method, a morphological-based method, a pixel-based method, an edge based method, model based method, a fuzzy clustering method, or k-means clustering.

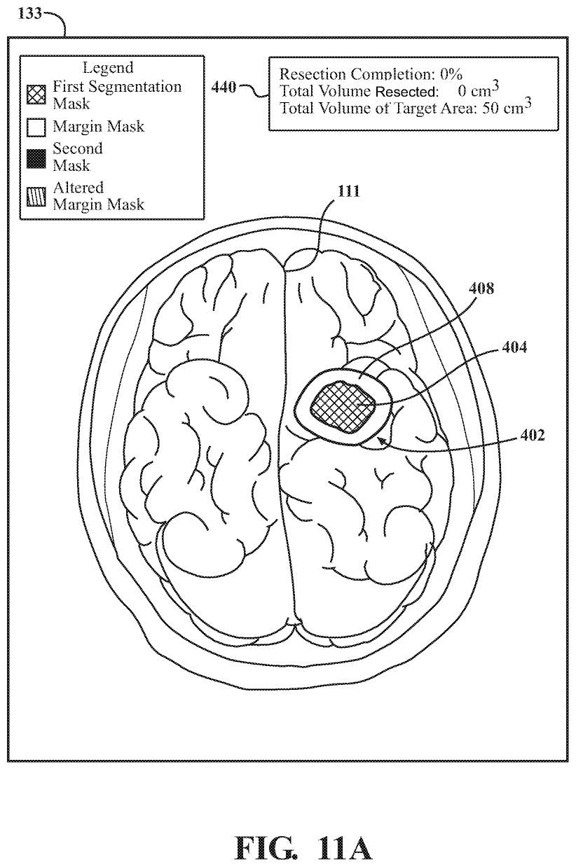

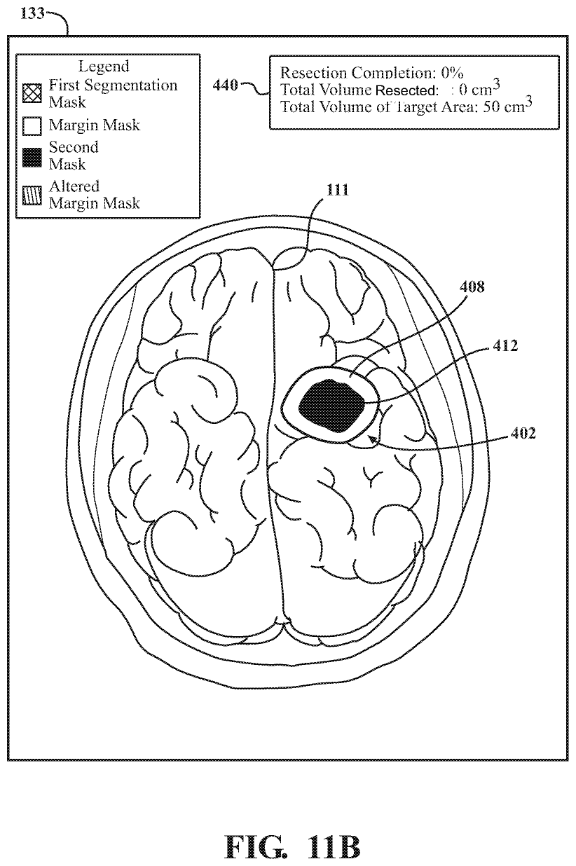

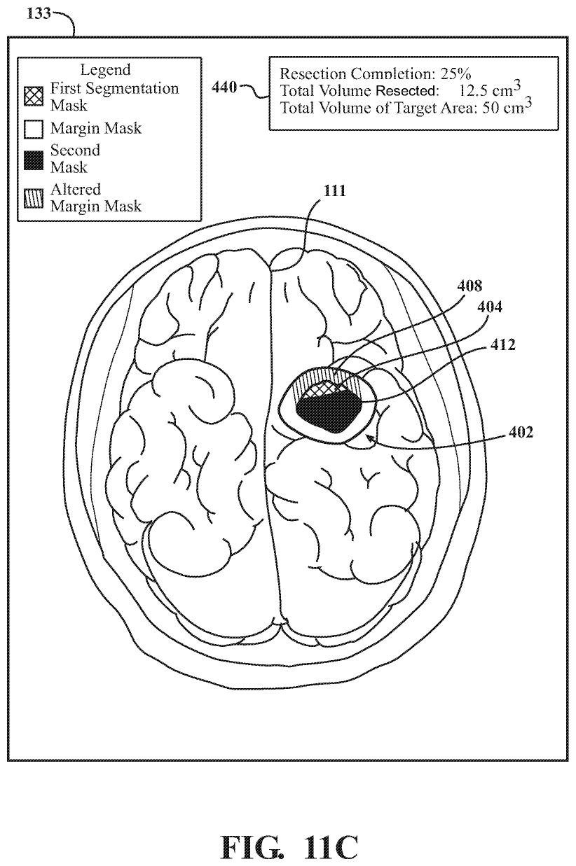

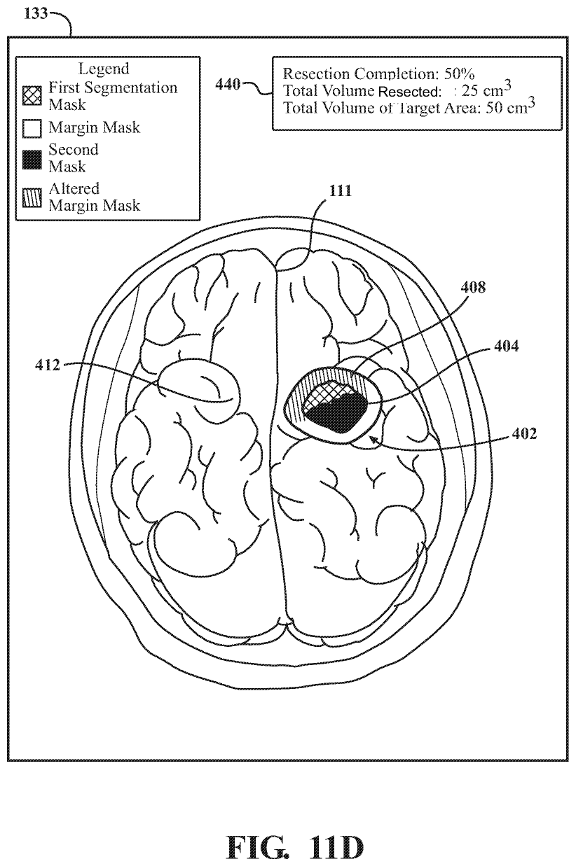

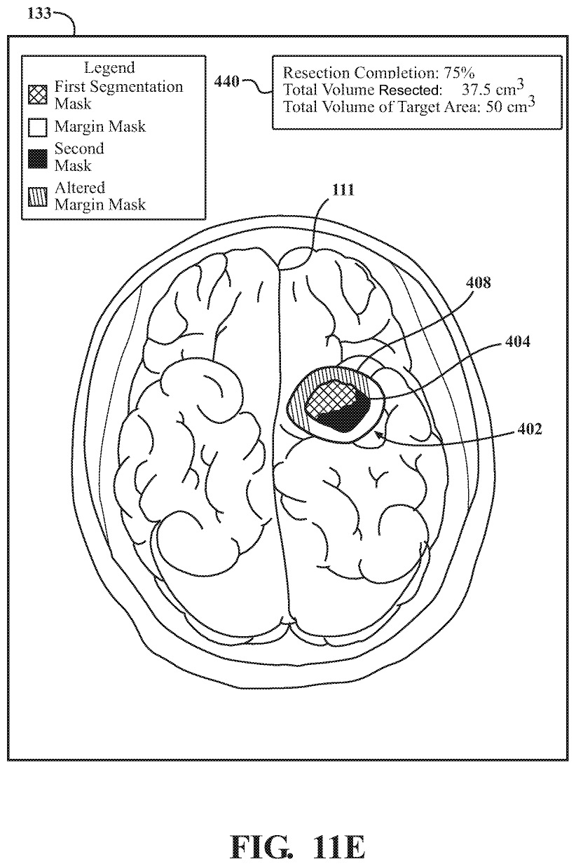

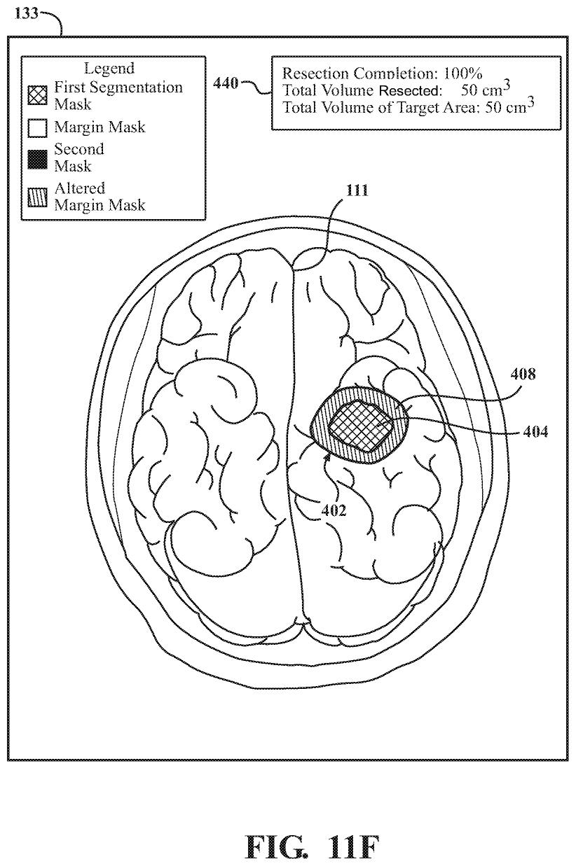

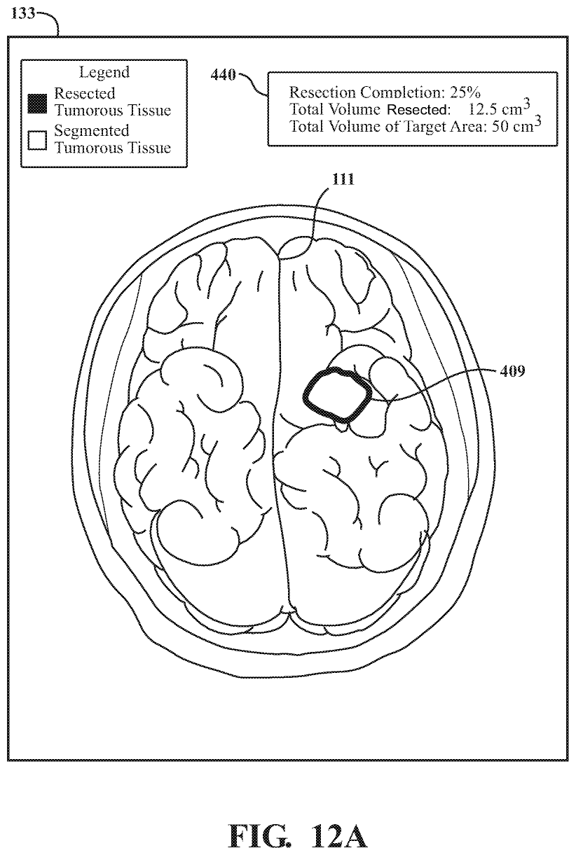

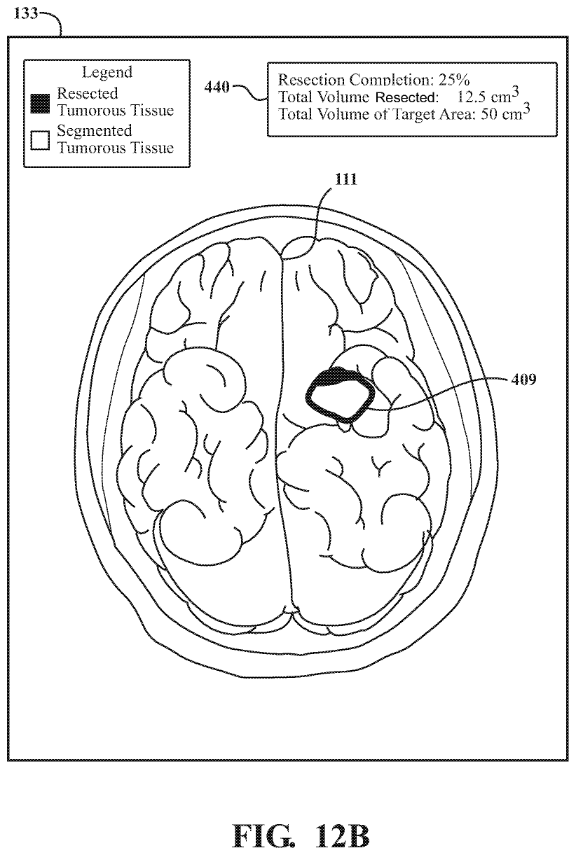

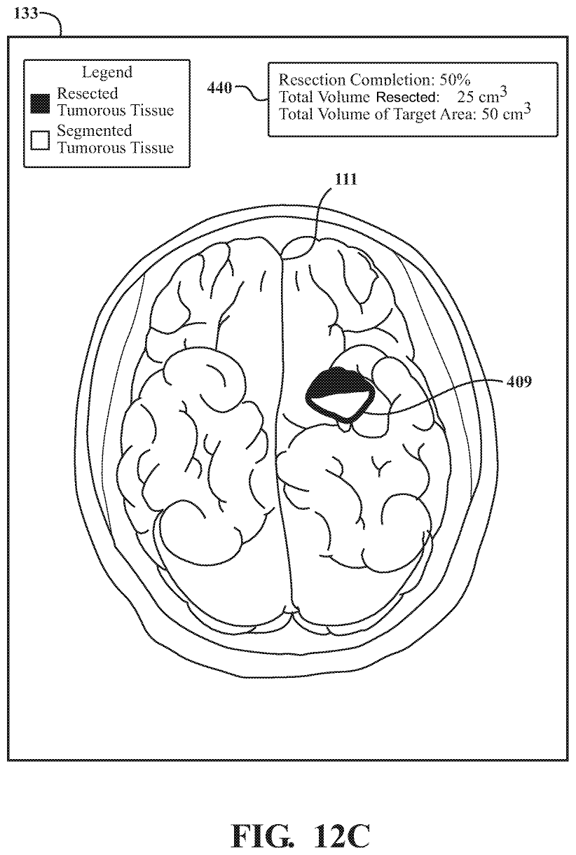

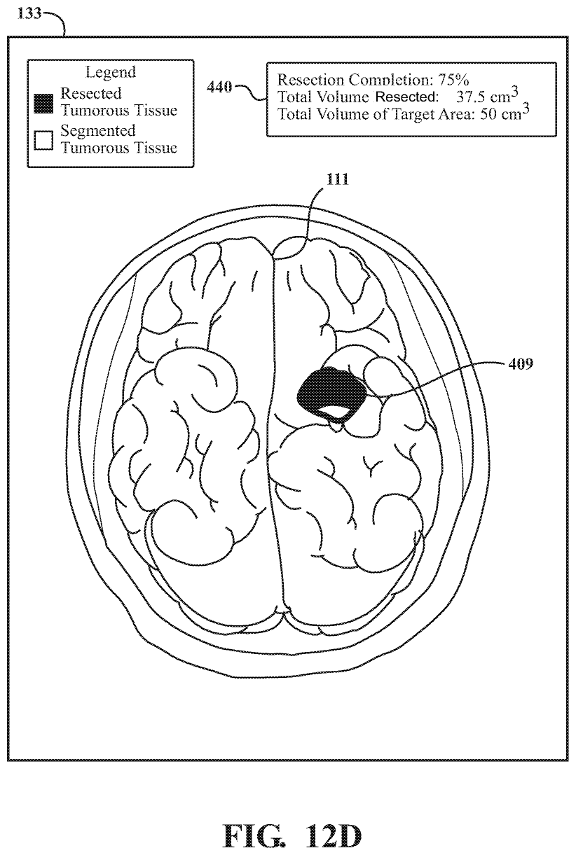

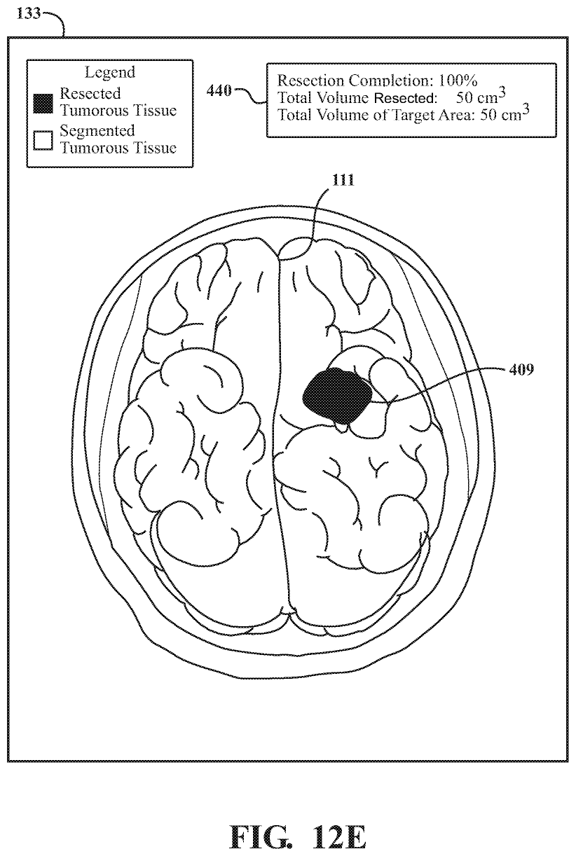

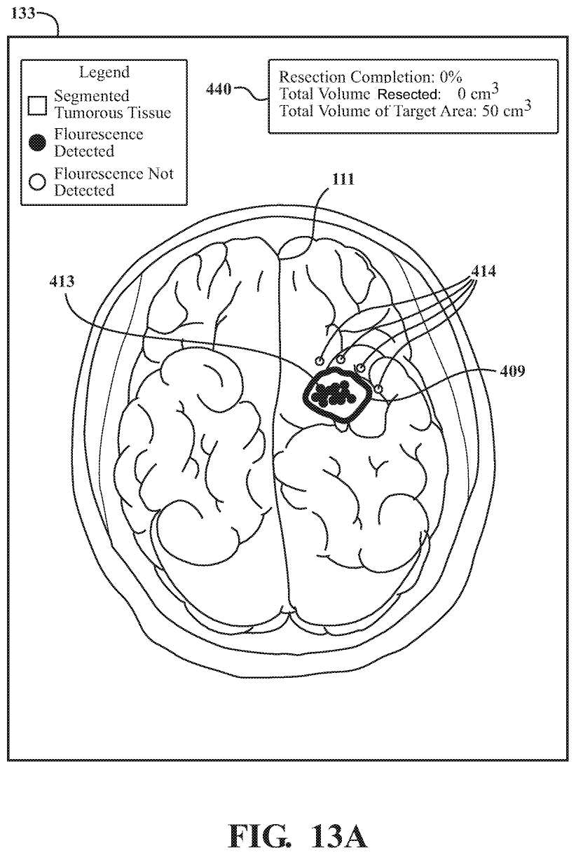

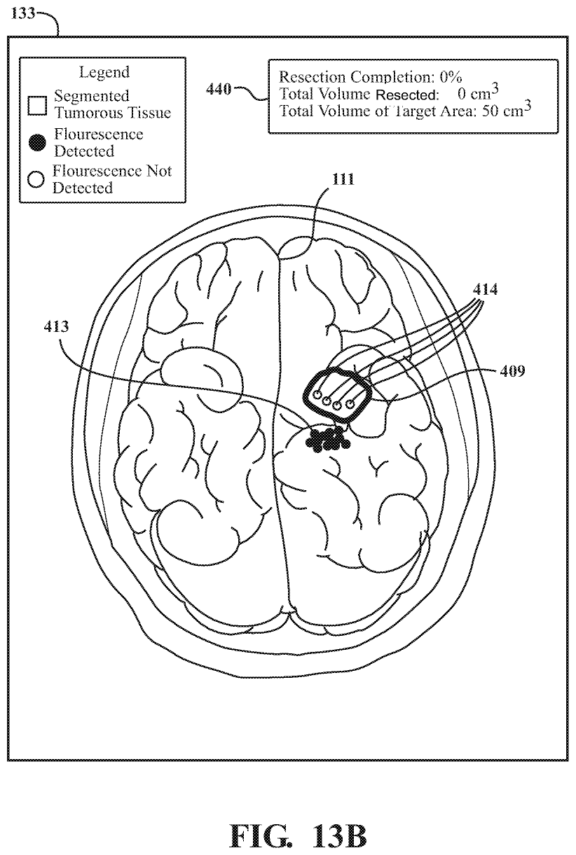

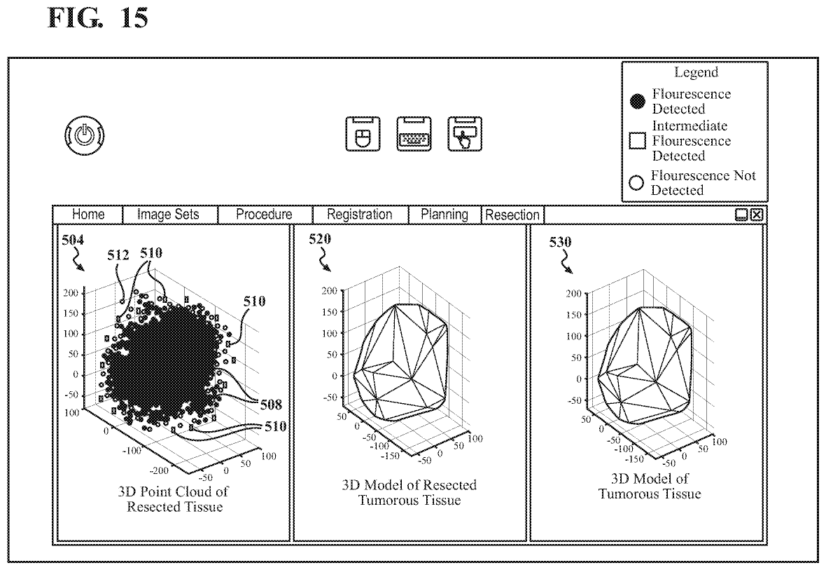

As will be described in FIGS. 11-14, the navigation computer 110 may display one or more indicators based on the level of fluorescence detected, a determination of tissue type and/or the pose of one or more of the surgical instruments to reflect a resection status of the tumorous tissue in real-time. The displayed resection status may be configured to alert a healthcare professional to any residual portion of the tumor. The indicators may be overlaid onto the images, 3D models or displayed by themselves. The indicators may by displayed in various different forms such as one or more masks overlaid onto the one or more images (as shown in FIGS. 11A-11F), 2D points with different shapes/patterns/colors (as shown in FIGS. 13A and 13B), 3D point cloud models with different shapes/patterns/colors (as shown in FIG. 15), and/or any other suitable graphic. The indicators may also include a modification of an existing graphic overlaid relative to the one or more images (as shown in FIGS. 11A-11F and FIGS. 12A-12E).

With particular reference to FIG. 11A, the axial view 133 of the GUI 131 is shown. During the segmentation, the navigation computer 110 may overlay a segmentation mask 404 onto the tumorous tissue to highlight the region of interest. Alternatively, the navigation computer 110 may draw or outline the tumorous tissue to highlight the region of interest. The navigation computer 110 may prompt the healthcare professional to provide input to indicate a margin around the tumorous tissue for resection. The margin is the plane along which a resection takes place and ideally it bisects healthy tissue around and outside the tumorous tissue. Based on the input provided by the healthcare professional, the navigation computer 110 may display a margin mask 408 representative of the margin around the segmentation mask 404. The margin in conjunction with the tumorous tissue may be referred to as the target area. The margin mask 408 may appear visually different than the segmentation mask 404 such as in a different color or different pattern than the segmentation mask 404. As shown in FIG. 11A, the margin mask 408 is shown with a white pattern (e.g., a first pattern).

The navigation computer 110 may be configured to generate one or more three-dimensional models (3D) of the brain, tumorous tissue, and or target area based on the images. A 3D model of the tumorous tissue may be reconstructed based on the segmented tumorous tissue of each of the 2D images processed from the 3D image. For example, once the tumor tissue has been segmented, the 2D images with the tumorous tissue can be reconstructed into the 3D model by placing the 2D images back into a sequence to provide the 3D model. Based on the reconstructed 3D model of the tumorous tissue, the navigation computer 110 may calculate the volume of the tumorous tissue or other parameters such as location within the brain, shape, etc. The navigation computer 110 may also be configured to include the margin selected by the healthcare professional in the 3D model. The navigation computer 110 may be configured to calculate one or more volume calculations of the target area including a volume of the tumorous tissue to be resected, a volume of the margin to be resected, and a total volume including volume of the tumorous tissue and volume of the margin to be resected. The calculations may be displayed relative to the images.

The navigation computer 110 may be configured to perform image or patient registration utilizing any suitable registration method to correlate the intra-operative pose of the patient with the images. The navigation computer 110 may employ an automatic image registration or a manual image registration method to perform the image or patient registration. For example, the navigation computer 110 may be configured to perform a point-based registration method. The navigation computer 110 may employ one of the registration methods described in U.S. Pat. No. 10,506,962 B2, the contents which are herein incorporated by reference. After the registration is performed, the pose of the suction tool 156 and/or the bipolar forceps 160 or other surgical tool may be displayed relative to the images.

As shown in FIG. 11B, prior to beginning the resection procedure, the navigation computer 110 may overlay a second mask 412 onto the segmentation mask 404. As shown in FIG. 11B, the second mask 412 is displayed over the entire portion of the initial segmentation mask 404. No portion of the initial segmentation mask 404 is visible when the second mask 412 is initially displayed prior to the resection of the target area 402 commencing. With reference to FIGS. 11C-11F, as the healthcare professional is resecting the target area 402, the navigation computer 110 may modify the margin mask 408 and the second mask 412 to reflect a resection status of the target area 402. For example, the navigation computer 110 may change a color or pattern of an area of the second mask 412 and/or margin mask 408 which corresponds to a portion of the target area 402 that has been resected. In another example, the navigation computer 110 may remove or delete the second mask 412 and/or the margin mask 408 as the healthcare professional resects the relevant tissue. Stated differently, as the healthcare professional resects the tumorous tissue, a portion of the second mask 412 that covers the corresponding portion of the tumorous tissue may be removed. Additionally, the navigation computer 110 may display a resection pane 440 that displays various calculations by the navigation computer 110 such as a total volume of the target area 402 or tumor resected, a total volume of the target area 402 or tumor remaining to be resected, and/or a degree of resection (e.g., a completion percentage), the latter of which may be determined based on one or more of the former calculations.

With particular reference to FIG. 11C, as the healthcare professional is performing the resection of the target area 402, the navigation computer 110 may update the resection pane 440 to reflect the various real-time calculations. In FIG. 11C, the resection pane 440 shows that the healthcare professional has resected 12.5 cm³ of the total target area of 50 cm³ which corresponds to a resection completion percentage of 25%. As shown, the navigation computer 110 has altered the target area 402 displayed on the screen to reflect the extent of the target area 402 removed. The navigation computer 110 has removed a portion of the second mask 412 associated with the portion of the tumorous tissue removed. The navigation computer 110 has also altered the margin mask 408 by changing a pattern of a portion of the margin mask 408 proportional to the amount of margin tissue that was removed by the healthcare professional.

With particular reference to FIG. 11D, as shown, the resection pane 440 indicates that the healthcare professional has resected 50% of the target area 402. As such, the navigation computer 110 has removed 50% of the second mask 412 and altered the margin mask 408 accordingly to reflect the portion of the target area 402 that has been removed. With reference to FIG. 11E, as shown in the resection pane 440, the healthcare professional has now removed 37.5 cm³ corresponding to 75% of the target area 402. As such, the navigation computer 110 has removed 75% of the second mask 412 and altered the margin mask 408 accordingly to reflect the portion of the target area 402 that has been removed.

With reference to FIG. 11F, the resection pane 440 indicates that the resection is complete at 100% when the total volume resected 50 cm³ is equal to the total volume of the target area 402 of 50 cm³. As shown, when the target area 402 has been completely resected, the navigation computer 110 no longer displays any portion of the second mask 412. Additionally, the navigation computer 110 may show the entire margin mask 408 as the altered margin mask to indicate that the entire margin area has been resected.

With reference to FIGS. 12A-12E, instead of generating the segmentation mask 404 as shown in FIGS. 11A-11F, as discussed above, the navigation computer 110 may generate an outline 409 around the tumorous tissue based on the one or more images. With reference to FIGS. 12B-12E, as the healthcare professional is resecting the tumorous tissue, the navigation computer 110 may be configured to fill in the area inside the outline 409 based on the pose of one or more of the surgical instruments (i.e., the suction tool 156 and/or the bipolar forceps 160), the level of fluorescence detected at each pose, and/or the determination of tissue type at each pose.

As shown in FIG. 12A, the healthcare professional has not yet commenced the resection procedure of the tumorous tissue indicated by the outline 409. As such, the outline 409 of the tumorous tissue is shown in an unfilled state and the resection pane 440 indicates the associated resection status of a resection completion at 0%, total volume resected at 0 cm³, and total volume of target area to be resected at 50 cm³. As shown in FIG. 12B, the outline 409 of the tumorous tissue is shown approximately 25% filled in based on the pose of one or more of the surgical instruments (i.e., the suction tool 156 and/or the bipolar forceps 160), the level of fluorescence detected at each pose, and/or the determination of tissue type at each pose. The resection pane 440, shows the resection completion at 25%, total volume resected at 12.5 cm³, and total volume of target area to be resected at 50 cm³. As shown in FIG. 12C, the outline 409 of the tumorous tissue is shown approximately 50% filled in based on the pose of one or more of the surgical instruments (i.e., the suction tool 156 and/or the bipolar forceps 160), the level of fluorescence detected at each pose, and/or the determination of tissue type at each pose. The resection pane 440 shows the resection completion at 50%, total volume resected at 25 cm³, and total volume of target area to be resected at 50 cm³. As shown in FIG. 12D, the outline 409 of the tumorous tissue is shown approximately 75% filled in based on the pose of one or more of the surgical instruments (i.e., the suction tool 156 and/or the bipolar forceps 160), the level of fluorescence detected at each pose, and/or the determination of tissue type at each pose. The resection pane 440 shows the resection completion at 75%, total volume resected at 37.5 cm³, and total volume of target area to be resected at 50 cm³. As shown in FIG. 12E, the outline 409 of the tumorous tissue is shown 100% filled in based on the pose of one or more of the surgical instruments (i.e., the suction tool 156 and/or the bipolar forceps 160), the level of fluorescence detected at each pose, and/or the determination of tissue type at each pose. The resection pane 440 shows the resection completion at 100%, total volume resected at 50 cm³, and total volume of target area to be resected at 50 cm³.

With reference to FIGS. 13A and 13B, similar to FIG. 12A-12E, the segmented tumorous tissue is indicated by the outline 409. As previously discussed during the resection procedure, brain shift may occur, causing the tumorous tissue to move from an initial registered pose. As such the pose of the tumorous tissue in the patient space may not correspond to the pose of the tumorous tissue in the image space. However, when the brain shift occurs, the healthcare professional has no way of knowing that the brain shift occurred by inspection of typical pre-operative images or intraoperative images captured before the brain shift occurred with the neurosurgical systems of the prior art. With the neurosurgical systems of the present disclosure, the navigation computer 110 may overlay one or more indicators relative to the images based on the pose of one or more of the surgical instruments (i.e., the suction tool 156 and/or the bipolar forceps 160), the level of fluorescence detected, and/or the determination of tissue type. The healthcare professional may inspect the one or more images with the indicators overlaid onto the images to make an assessment as to how much brain shift may have occurred and whether additional inter-operative imaging is warranted. With particular reference to FIG. 13A, the indicators are shown as point by point indicators 413, 414. In FIG. 13A, the navigation computer 110 overlays the first point by point indicators 413 to indicate where fluorescence, or more particularly fluorescence corresponding to a given type of tissue (e.g., target or tumorous tissue), has been detected. The first point by point indicators 413 are shown displayed in a solid color. The navigation computer 110 overlays the second point by point indicators 414 onto the one or more images to indicate where fluorescence, or more particularly fluorescent corresponding to the given type of tissue (e.g., target or tumorous tissue), has not been detected. The second point by point indicators 414 are shown by unfilled circles. As one can see from FIG. 13A, the first point by point indicators 413 are all within the outline 409 of the segmented tumorous tissue and the second point by point indicators are all outside of the outline 409 of the segmented tumorous tissue. As such, the healthcare professional may make the determination that no brain shift has occurred or a nominal amount of brain shift has occurred. With reference to FIG. 13B, the first point by point indicators 413 (i.e., the points indicating that fluorescence was detected) are shown outside of the outline 409 of the segmented tumorous tissue and the second point by point indicators 414 (i.e., the points indicating that fluorescence was not detected) are within the outline 409 of the segmented tumorous tissue. As such, the healthcare professional may make the determination that substantial brain shift has occurred. The healthcare professional may choose to perform additional intra-operative imaging in some instances when it is determined that substantial brain shift has occurred in order to reassess the tumorous tissue relative to the healthy tissue.

The navigation computer 110 may be configured to store as resection data poses of the surgical instruments (i.e., the suction tool 156 and/or the bipolar forceps 160) relative to a target area including tumorous tissue, along with an associated determination of whether or not tissue associated with the respective poses was tumorous tissue or healthy tissue based on collected fluorescence emitted from the tissue. Once the navigation computer 110 has collected enough resection data, the navigation computer 110 may use the resection data to account for brain shift. More specifically, once there is enough resection data collected, the navigation computer 110 may be configured to match the resection data to a shape or contour of a portion of the tumorous tissue. With reference to FIG. 13, the tumorous tissue indicated by the segmentation mask 404 may include one or more unique portions defined by a distinctive shape. The navigation computer 110 may use the unique portions to derive one or more transform functions for the distinctive shape between the patient space and the image space. The one or more derived transform functions may than be used by the navigation computer 110 to extrapolate an updated pose of the tumorous tissue relative to the images due to brain shift. In this manner, the system according to the present disclosure is able to help account for brain shift occurring during the resection procedure.

Based on the known spatial relationships between the tumorous tissue and important/eloquent structures such as, sensorimotor cortex, language cortex, subcortical structures (e.g., basal ganglia and internal capsule) gathered from the images, the navigation computer 110 may derive one or more transform functions for determining a revised pose of one or more eloquent structures effected by the brain shift. In an example, the navigation computer 110 may be configured to derive transform functions for eloquent structures within a threshold range of the tumorous tissue. Stated differently in the example provided, the navigation computer 110 may be configured to derive transform functions for eloquent structures at the greatest risk to be impacted during the resection procedure.

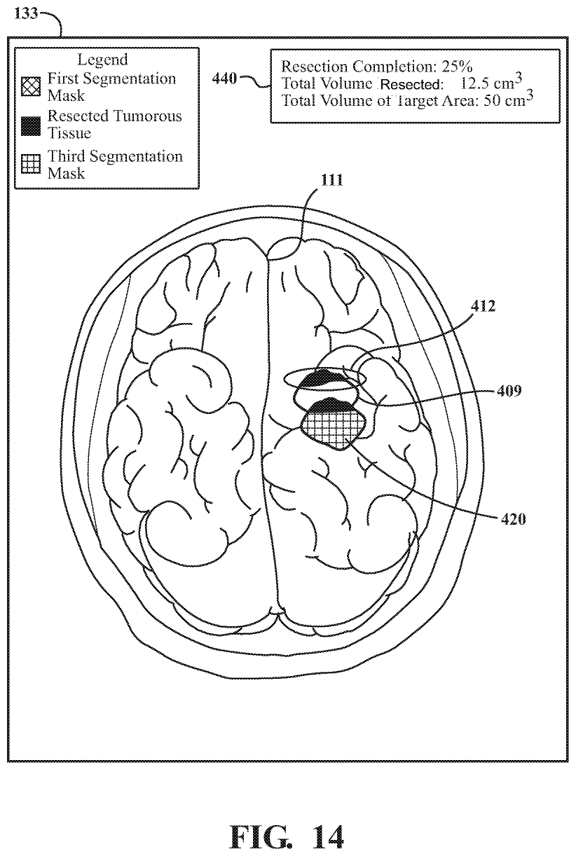

With reference to FIG. 14, once the navigation computer 110 has derived a transform function for a unique portion of the tumorous tissue, the navigation computer 110 may be configured to calculate a revised pose of the tumorous tissue and/or the one or more of the eloquent structures. As shown in FIG. 14, the navigation computer 110 overlays a third mask 420 onto the one or more images which is representative of the calculated revised pose of the tumorous tissue. Although not shown in FIG. 14, the navigation computer 110 may also be configured to overlay one or more graphics representative of the calculated revised pose of the eloquent structures at the greatest risk to be impacted during the resection procedure.

With reference to FIG. 15, the navigation computer 110 may be configured to display graphical user interface (GUI) 137 including a 3D point cloud 504 including measured fluorescence intensity of PpIX of the resected tissue, a 3D model of the resected tumorous tissue 520, and a 3D model of the tumorous tissue 530. The 3D point cloud may be generated based on the pose of the surgical instruments (i.e., the suction tool 156 and/or the bipolar forceps 160) and the level of fluorescence detected. The navigation computer 110 may plot the various points with varying levels of RGB (red, green, blue) graphics to represent the various levels of fluorescence detected. For example, the navigation computer 110 may plot a red point to indicate that fluorescence intensity of the PpIX is greater than or equal to a first threshold (i.e., the tissue corresponds to the tumorous tissue), green to indicate that fluorescence has not been detected or that the fluorescence intensity of the PpIX is below a second threshold (i.e., the tissue corresponds to healthy tissue) or other colors to indicate that the fluorescence intensity of the PpIX is less than the first threshold but greater than or equal to the second threshold (i.e., the tissue type cannot be readily determined from the fluorescence levels). The 3D point cloud 504 with varying levels of RGB indicators may better help a healthcare professional understand the heterogeneity of the tumorous tissue. The 3D point clouds 504 may also be generated with various shapes/patterns to represent the varying levels of fluorescence intensity of the PpIX. The healthcare professional may be able to gather additional knowledge as to where the most aggressive and most cancerous cells are present and where the tumorous tissue is most likely to occur based on the 3D point cloud 504.

In FIG. 15, in the 3D point cloud 504, the solid circles 508 indicate that fluorescence intensity of the PpIX is greater than or equal to the first threshold, the hollow circles 512 indicate fluorescence has not been detected or that the fluorescence intensity of the PpIX is below the second threshold, and the squares 510 to indicate the fluorescence intensity of the PpIX is less than the first threshold but greater than or equal to the second threshold (i.e., an intermediate level of fluorescence has been detected). Based on the point cloud data, the navigation computer 110 may generate a 3D model of the resected tumorous tissue 520. For example, the navigation computer 110 may generate a convex hull based on the 3D point cloud 504, in particular, the 3D point cloud data where the fluorescence intensity of the PpIX is greater than or equal to the first threshold. The health care professional may compare the shape of the 3D model of the resected tumorous tissue to the shape of the 3D model of the tumorous tissue 530 to make an assessment of the resection status.

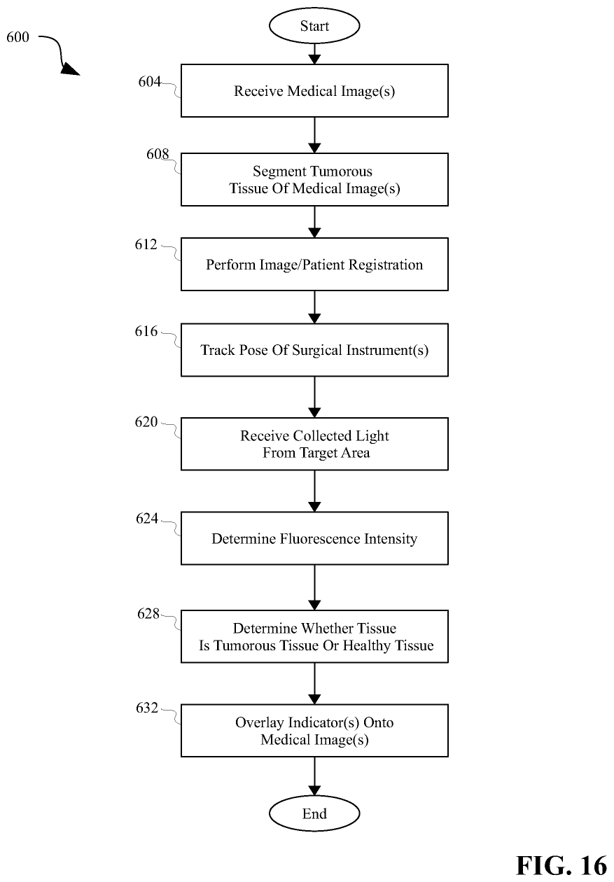

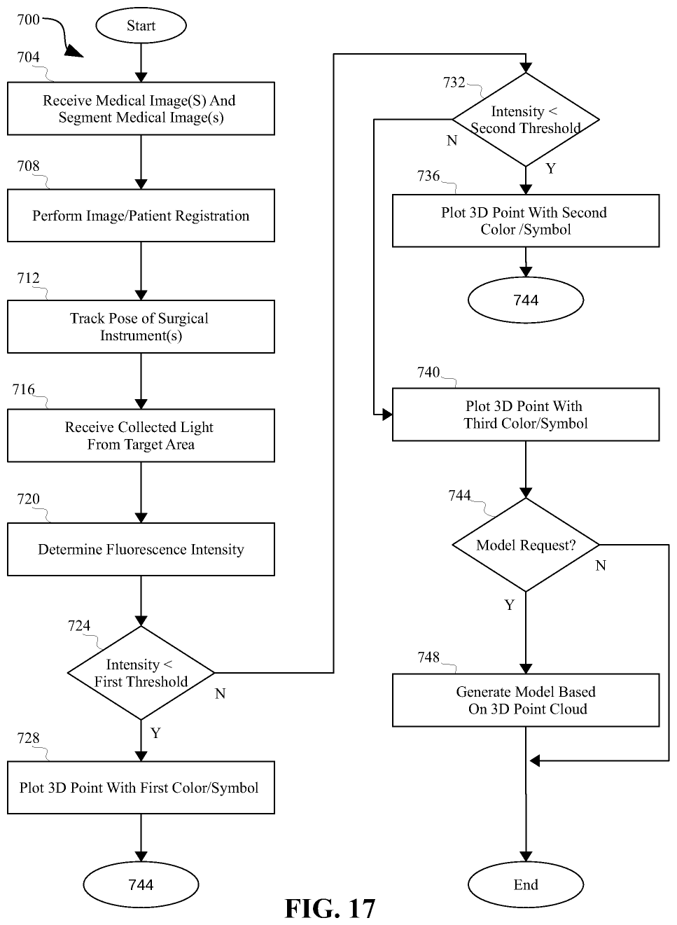

Referring to FIGS. 16 and 17, two flowcharts illustrating methods implemented by the neurosurgical system 100 are shown. As will be appreciated from the subsequent description below, the methods merely represent exemplary and non-limiting flowcharts that describe particular methods for implemented by the neurosurgical system 100. The methods are in no way intended to serve as complete methods or catchall methods implemented by the neurosurgical system 100. Although the methods are illustrated as ending, the method may return to start and be performed as a continuous-loop.

With particular reference to FIG. 16, method 600 is depicted. At 604, the method 600 may receive one or more medical images. At 608, the method 600 may segment the tumorous tissue of the medical image(s). At 612, the method 600 may perform image/patient registration. At 612, the method 600 may track a pose of one or more of the surgical instruments(s), such as the suction tool 156 or the bipolar forceps 160. At 620, the method 600 may receive collected light from the target area 402. At 624, the method may determine fluorescence intensity. At 628, the method 600 may determine whether tissue correspond to tumorous tissue or healthy tissue based on the fluorescence intensity. At 632, the method 600 may overlay one or more indicator(s) onto the one or more medical image(s) and the method may end or continue back at 604.

With particular reference to FIG. 17, method 700 is depicted. At 704, the method 700 may receive one or more medical images and segment the tumorous tissue of the medical image(s). At 708, the method 700 may perform image/patient registration. At 712, the method 700 may track a pose of one or more of the surgical instruments(s), such as the suction tool 156 or the bipolar forceps 160. At 716, the method 700 may receive collected light from the target area. At 720, the method may determine fluorescence intensity. At 724, the method 700 may determine if the fluorescence intensity is less than a first threshold. If so, the method 700 may continue at 728; otherwise, the method 700 continues to 732. At 728, the method 700 may plot the 3D point based on a pose of one of the surgical instruments(s) with a first color and/or first symbol and continue to 744. At 744, the method 700 determines whether input from the healthcare professional has been received for a model. If so, the method 700 may continue to 748 where the method 700 generates a model based on the 3D point cloud. If no input is received from the healthcare professional to generate a model, the method 700 may end or continue back at 704.

At 732, the method 700 may determine if the fluorescence intensity is less than a second threshold. If so, the method 700 may continue at 736; otherwise, the method 700 continues to 740. At 736, the method 700 may plot the 3D point based on a pose of one of the surgical instruments(s) with a second color and/or a second symbol and the method 700 may continue to 744. At 740, the method 700 may plot the 3D point based on a pose of one of the surgical instruments(s) with a third color and/or a third symbol and the method may continue to 744.

Although several of the above-described functions, features and processes are described in reference to a brain procedure, it will be appreciated such functions, features and processes may also be applied to other human organs. In other words, the navigation computer 110 may be configured to track a resection status and/or account for organ shift during a resection procedure involving the alternative organ as described above.

The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the examples is described above as having certain features, any one or more of those features described with respect to any example of the disclosure can be implemented in and/or combined with features of any of the other examples, even if that combination is not explicitly described. In other words, the described examples are not mutually exclusive, and permutations of one or more examples with one another remain within the scope of this disclosure.

Spatial and functional relationships between elements (for example, between controllers, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements.

As used herein, the phrase at least one of A, B, and C should be construed to mean a logical (A OR B OR C), using a non-exclusive logical OR, and should not be construed to mean “at least one of A, at least one of B, and at least one of C.” The term subset does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “controller” or “module” may be replaced with the term “circuit.” The term “controller” may refer to, be part of, or include: an Application Specific Integrated Circuit (ASIC); a programmable system on a chip (PSoC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The controller may include one or more interface circuits with one or more transceivers. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The controller may communicate with other controllers using the interface circuit(s). Although the controller may be depicted in the present disclosure as logically communicating directly with other controllers, in various implementations the controller may actually communicate via a communications system. The communications system may include physical and/or virtual networking equipment such as hubs, switches, routers, gateways and transceivers. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the controller may be distributed among multiple controllers that are connected via the communications system. For example, multiple controllers may implement the same functionality distributed by a load balancing system. In a further example, the functionality of the controller may be split between a server (also known as remote, or cloud) controller and a client (or user) controller.

Some or all hardware features of a controller may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a controller may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. The term shared processor circuit encompasses a single processor circuit that executes some or all code from multiple controllers. The term group processor circuit encompasses a processor circuit that, in combination with additional processor circuits, executes some or all code from one or more controllers. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple controllers. The term group memory circuit encompasses a memory circuit that, in combination with additional memories, stores some or all code from one or more controllers.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above may serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

1. A neurosurgical method for determining a resection status during a tumor resection procedure, the method comprising:
acquiring at least one medical image of a human organ including a tumor;
determining a pose of a suction tool, including at least one optical fiber and a navigation tracker, relative to a target area of the human organ based on the navigation tracker, the target area including the tumor and a margin area surrounding the tumor;
generating excitation light for the at least one optical fiber to excite the target area of the human organ;
receiving, from the at least one optical fiber, collected fluorescence emitted from the target area and associated with the pose of the suction tool;
determining whether tissue in the target area and associated with the pose of the suction tool corresponds to the tumor based on the collected fluorescence associated with the pose of the suction tool; and
displaying a resection status of the target area relative to the at least one medical image based on the determination of whether the tissue corresponds to the tumor and the pose of the suction tool.

2. The neurosurgical method of claim 1, wherein displaying the resection status includes alerting a healthcare professional to any residual portion of the tumor.

3. The neurosurgical method of claim 1, wherein displaying the resection status includes overlaying a first indicator onto the at least one medical image of the human organ based on the pose of the suction tool in response to determining that the tissue corresponds to the tumor, and overlaying a second indicator onto the at least one medical image of the human organ based on the pose of the suction tool in response to determining that the tissue does not correspond to the tumor.

4. The neurosurgical method of claim 3, further comprising generating a 3D model of a brain, wherein the first indicator and the second indicator are displayed as point cloud models relative to the 3D model.

5. The neurosurgical method of claim 1, further comprising storing as resection data the pose of the suction tool and an associated determination of whether the tissue is tumorous or not.

6. The neurosurgical method of claim 5, further comprising calculating, based on the resection data, at least one of a volume of tissue remaining that corresponds to the tumor and a volume of tissue resected that corresponds to the tumor.

7. The neurosurgical method of claim 6, further comprising calculating a degree of resection based on the at least one of the volume of tissue remaining that corresponds to the tumor and the volume of tissue resected that corresponds to the tumor.

8. The neurosurgical method of claim 1, further comprising storing as resection data a property of collected fluorescence emitted from the target area for each of a plurality of stored poses of the suction tool relative to the target area and an associated determination of whether tissue associated with the stored pose corresponds to the tumor.

9. The neurosurgical method of claim 1, wherein the human organ is a brain, the method further comprising accounting for a shift of at least a portion of the brain occurring during the resection procedure.

10. The neurosurgical method of claim 9, wherein accounting for the shift of at least a portion of the brain includes predicting a pose of the tumor relative to the at least one medical image based on resection data indicating, for each of a plurality of stored poses of the suction tool relative to the target area, an associated determination of whether tissue of the target area associated with the stored pose corresponds to the tumor based on collected fluorescence emitted from the tissue.

11. The neurosurgical method of claim 10, wherein predicting the pose of the tumor includes:
matching a portion of the resection data to a portion of a contour of the tumor; and
extrapolating the pose of the tumor relative to the at least one medical image based on the portion of the resection data that has been matched to the portion of the contour of the tumor.

12. A neurosurgical system for determining a resection status during a tumor resection procedure, the neurosurgical system comprising:
a suction tool configured to apply suction to a target area of a patient, the target area including tumorous tissue, and the suction tool including a suction cannula defining a lumen, and the suction tool including a navigation tracker;
an optical fiber, coupled to the suction cannula, the optical fiber being configured to collect a fluorescence emitted from the target area;
an excitation source configured to emit an excitation light, the excitation light having a wavelength to induce the fluorescence emitted from the target area;
an optical instrument coupled to the optical fiber, the optical instrument configured to convert the fluorescence emitted from the target area and collected by the optical fiber into an electrical signal; and
a surgical navigation system configured to:
receive at least one medical image of a human organ including a tumor corresponding to the tumorous tissue of the target area;
determine a pose of the suction tool relative to the target area based on the navigation tracker;
determine whether tissue in the target area corresponds to the tumorous tissue based on collected fluorescence associated with the pose of the suction tool; and
display at least one indicator relative to the at least one medical image based on the determination of whether the tissue in the target area corresponds to the tumorous tissue and the pose of the suction tool.

13. The neurosurgical system of claim 12, wherein the at least one indicator alerts a healthcare professional to any residual portion of the tumorous tissue.

14. The neurosurgical system of claim 12, wherein the surgical navigation system is configured to overlay a first indicator onto the at least one medical image of the human organ based on the pose of the suction tool in response to determining that the tissue corresponds to the tumorous tissue, and overlay a second indicator onto the at least one medical image of the human organ based on the pose of the suction tool in in response to determining that the tissue does not correspond to the tumorous tissue.

15. The neurosurgical system of claim 14, wherein the first indicator and the second indicator are displayed as point cloud models relative to a 3D model of the human organ.

16. The neurosurgical system of claim 12, wherein the surgical navigation system is configured to store as resection data the pose of the suction tool and an associated determination of whether the tissue associated with the pose of the suction tool is tumorous or not.

17. The neurosurgical system of claim 16, wherein the surgical navigation system is configured to calculate, based on the resection data, at least one of a volume of tissue remaining that corresponds to the tumorous tissue and a volume of tissue resected that corresponds to the tumorous tissue.

18. The neurosurgical system of claim 17, wherein the surgical navigation system is configured to calculate a degree of resection based on the at least one of the volume of tissue remaining that corresponds to the tumorous tissue and the volume of tissue resected that corresponds to the tumorous tissue.

19. The neurosurgical system of claim 12, wherein the human organ is a brain and the surgical navigation system is configured to account for a shift of at least a portion of the brain occurring during the resection procedure.

20. The neurosurgical system of claim 19, wherein the surgical navigation system is configured to account for the shift of at least a portion of the brain by predicting a pose of the tumorous tissue relative to the at least one medical image based on resection data indicating, for each of a plurality of stored poses of the suction tool, an associated determination of whether tissue associated with the stored pose corresponds to the tumorous tissue based on collected fluorescence emitted from the tissue.

21-29. (canceled)
